(19) United States
(12) Patent Application Publication
(10) Pub. No.: US 2005/0177891 A1
(43) Pub. Date: Aug. 11, 2005

(54) Novel plant glucosidase I and use thereof for producing recombinant proteins with modified glycosylation

(76) Inventors: Murielle Boisson, Anse (FR); Veronique Gomord, Rouen (FR); Patrice LeRouge, Grand Couronne (FR); Loic Faye, Saint Jacques Sur Darnetal (FR); Michel Caboche, Maurepas (FR); Loic Lepiniec, Bures sur Yvette (FR)

Correspondence Address: JACOBSON HOLMAN PLLC, 400 SEVENTH STREET N.W., SUITE 600, WASHINGTON, DC, 20004, US

(21) Appl. No.: 10/257,967

(22) PCT Filed: Apr. 25, 2001
(86) PCT No.: PCT/FR01/01266

(30) Foreign Application Priority Data
Apr. 26, 2000 (FR) 00/05317

(51) Int. Cl. C12N 15/82; A01H 5/00; C12Q 1/68; G01N 33/53

(52) U.S. Cl. 800/278; 536/24.3; 435/320.1; 435/252.3; 435/419; 435/430; 800/294; 800/298; 800/285; 435/6; 530/350; 435/7.1

(57) ABSTRACT

The invention concerns a novel plant glucosidase I and nucleic acids coding for said enzyme involved in N-glycosylation of proteins during translation. The invention also concerns means for detecting said protein and nucleic acids, such as antibodies or nucleotide probes and primers.

This invention relates to a novel plant glucosidase I and nucleic acids coding for said enzyme involved in the N-glycosylation of proteins being translated. It also relates to detecting means for such protein and nucleic acids, such as antibodies or also probes and nucleotide primers.

The invention also relates to recombinant vectors comprising a nucleic acid coding for the novel plant glucosidase I, having host cells transformed by a nucleic acid or a recombinant vector according to the invention, as well as to transgenic plants having some of or all their cells being transformed with a nucleic acid or a recombinant vector according to the invention.

The invention also relates to means adapted to increase or, on the contrary, to inhibit the expression of a nucleic acid coding for the novel glucosidase I in plants, with the aim of producing proteins, more particularly recombinant proteins the glycosylation of which is modified.

In plants, as in all the eukaryotes, N-glycosylation consists in a covalent fixation of a more or less complex oligosaccharide onto a protein, at the level of a glycosylation site consisting of the NXS/T aminoacid sequence, wherein X represents any one of aminoacids, except proline (P) and aspartic acid (D).

The N-glycosylation process starts in the endoplasmic reticulum with the transfer of a precursor oligosaccharide of the (Glc₃ Man₉ GlcNAc₂) type on the N residue in a glycosylation site of the protein being translated.

Such a precursor oligosaccharide is subjected to modifications in the endoplasmic reticulum. Firstly, the glucosidase I cleaves a glucose and the glucosidase II cleaves the next two glucoses, the oligosaccharide fixed onto the glycosylation site having then the form (Man₉ GlcNAc₂).

Secondly, the protein is transferred into the Golgi apparatus within which the oligosaccharide is going to undergo a great deal of additional modifications. More particularly, α-1,3 fucose and β-1,2 xylose residues may be added. The α-1,3 fucose and β-1,2 xylose residues are specific to plants and seem to be present in molluscs, insects and nematodes; they have never been identified in mammals.

The glycans fixed onto the protein glycosylation sites play an important part in several mechanisms. They allow more particularly the protein structure to be maintained in a biologically active conformation, they ensure a protection for the peptidic chain against the attack by proteolytic enzymes and may interfere in the cell recognition phenomena (Weil et al., 1989).

The glucosidase I is also referred to as mannosyl-oligosaccharide glucosidase, having the international class EC : 3.2.1.106.

The glucosidase I is a type II membrane enzyme of the endoplasmic reticulum, the C-terminal end being located in the lumen of the endoplasmic reticulum.

Such an enzyme catalyzes the first step of the N-glycosylation process by hydrolyzing the terminal glucose residue of the precursor oligosaccharide. This protein has been purified and characterized in several species.

In yeast, the glucosidase I has a 95 kDa size (Bause et al., 1986). The glucosidase I has also been isolated and characterized from pig liver (Bause et al., 1989), in the bovine mammary gland where it seems to be present in the form of a tetramer (Schailubhai et al., 1987) and from calf liver (Hettkamp et al., 1984). In these three species, the glucosidase I has a 85 kDa size and the similarities in their respective aminoacid sequences show that such a protein is rather well preserved in the course of evolution (Pukkazhenti et al., 1993).

A human cDNA with 2 881 pb coding for a glucosidase I has also been isolated (Kalz-Füller et al., 1995). The analysis of the aminoacid sequence derived from the cDNA sequence has evidenced a potential glycosylation site on the aminoacid in a position 655, the presence of a highly hydrophobic region corresponding to the putative transmembrane region from the aminoacid in a position 38 to the aminoacid in a position 58 towards the N-terminal end and in the presence of a cytosol region with 37 aminoacids at the N-terminal end as well as an extended C-terminal region located in the lumen of the endoplasmic reticulum. This is a membrane protein of the type II.

In plants, the glucosidase I has been isolated from young bean plants (Mung Bean) by (Szumilo et al. 1986), such an enzyme having been studied in details by Zeng et al. in 1998. This glucosidase I has a 97 kDa size and can be distinguished from the animal glucosidases I through a different sensitivity to aminoacid modifying agents.

For example, an histidine modifying agent inactivates the bean glucosidase I but not that of pig liver, whereas a cystein modifying agent only activate the glucosidase I from pig liver.

Whether they come from a yeast, a mammal or a plant, the known glucosidases I share some common features: it is a α-1,2-glucosidase the catalytic activity of which does not require the presence of a metal ion and which is N-glycosylated on a single site.

Until now, no genomic DNA or messenger RNA coding for a plant glucosidase I has ever been isolated and characterized.

However, there is nowadays a very important need for means making it possible to produce polypeptides of industrial interest in plants, more particularly recombinant proteins, and having a modified glycosylation.

More particularly, there is an increasing need for proteins produced in plants, which do not have the allergenic features imparted by the fucose and xylose residues being contained in the complex oligosaccharides fixed at the level of the glycosylation sites in proteins from plant origin.

There is also an increasing need for proteins coming from allergenic effect free cereals or also from recombinant proteins of dietary or therapeutic interest which do not generate allergenic phenomenon in the consumer or the patient.

With a view to modifying the nature of the N-glycosylafion in plants, it has been suggested to stop the cascade of glycosylation enzyme reactions, before the glycan maturation steps, or alternatively, to get the plants to produce, by transgenesis, complex oligosaccharides of an animal type (Chrispeels and Faye, 1998).

It has also been contemplated to use castanospermine, which is a glucosidase I inhibiting agent, more particularly in plant cell cultures in a fermentor, so as to synthesize proteins carrying a N-glycan having a structure close to that of the precursor common to all the eukaryotes.

The invention has made it possible to isolate and to characterize for the first time the transcription product of a gene coding for a plant glucosidase I catalyzing the cleavage of the first external glucose residue of the (Glc₃ Man₉ Glc Nac₂) precursor oligosaccharide and thereby to make available to the man skilled in the art means allowing to modulate the expression of the corresponding gene in a plant and in particular to inhibit or to block the translation of the glucosidase I in a plant, so as to produce in that plant glycosylated proteins having no allergenic and/or immunogenic osidic residues, such as fucose and xylose residues.

The Applicant has thus isolated and characterized a complementary DNA corresponding to the messenger RNA coding for a glucosidase I in Arabidopsis thaliana, the corresponding gene being designated as AtGCS1.

The Applicant has also shown that the interruption of the genomic sequence of the AtGCS1 gene in a plant leads to the production of proteins with a modificed glycosylation.

More particularly, it has been shown that the blocking of the expression of the AtGCS1 gene according to the invention led to the production of proteins having their glycosylation sites being occupied by precursor oligosaccharides and not by mature N-glycans. The analysis of the proteins produced by plants having the expression of the AtGCS1 gene being blocked has made it possible to determine a total absence of allergenic xylose and fucose residues. The blocking of the expression of the AtGCS1 plant glucosidase I gene causes the seed development to stop.

A DNA-T insertion mutant affected in the protein N-glycosylation has been isolated. The DNA-T is inserted in a gene localized in the BAC T1F15 coming from the DNA bank of Arabidopsis thaliana TAMU of the Columbia ecotype which has been recorded in the GenBank database under the access number AC004393.

The recorded sequence in the above-mentioned database has been shown as being similar to the human glucosidase I.

The genomic sequence of the AtGCS1 gene also shows a strong similarity (66% identity in nucleic acids) with a sequence of BAC F316 in the IGF databank recorded in the GenBank database under the access number AC002396.

The sequence contained in the BAC F316 has also been shown as being similar to the human glucosidase I. Homologies between the AtGCS1 gene coding sequence has been found, for example, with mouse, rat and man glucosidase I (38% identity in nucleic acids) of a putative protein of C. elegans (36% identity in nucleic acids) or of Schizzo.pombe (31% identity in nucleic acids) or also of yeast (28% identity in nucleic acids).

The sequence of the gene described in the GenBank database under the access number AC004353 comprises 20 exons and 19 introns. According to such an analysis, the sequence of the gene contained in BAC T1F15 would be transcribed into a messenger RNA coding for a putative protein with a 864 aminoacid length.

The AtGCS1 gene according to the invention, which comprises 22 exons and 21 introns, allows for the synthesis of a messenger RNA coding for a glucosidase I with a 852 aminoacid length.

Therefore, a first object of the invention aims at a nucleic acid comprising at least 20 consecutive nucleotides of a polynucleotide coding for a glucosidase I with the aminoacid sequence SEQ ID N^{o} 1 or a nucleic acid with a complementary sequence.

Preferably, a nucleic acid according to the invention has an isolated and/or purified form.

The term “isolated”, as used herein, refers to a biological material (nucleic acid or protein) which has been substracted from its original environment (the environment in which it is naturally localized).

For example, a polynucleotide present in the natural state in a plant or an animal is not isolated. The same polynucleotide separated from adjacent nucleic acids within which it is naturally inserted in the plant or animal genome is considered as being “isolated”.

Such a polynucleotide may be included into a vector and/or such a polynucleotide may be included into a composition and nevertheless remain in an isolated state as the vector or the composition does not represent its natural environment.

The term “purified” does not require that the material be present in an absolute purity form, excluding the presence of other compounds. It is rather a relative definition.

A polynucleotide is in the “purified” state after purification of the starting material or the natural material of at least one magnitude order, preferably 2 or 3 and more preferably 4 or 5 magnitude orders.

As used in the present invention, the expression “nucleotidic sequence” can be used to indiscriminately refer to a polynucleotide or a nucleic acid. The expression “nucleotidic sequence” encompasses the genetic material as such and is therefore not limited to the information regarding the sequence thereof.

The expressions “nucleic acid”, “polynucleotide”, “oligonucleotide” or also “nucleotidic sequence” encompass RNA, DNA, cDNA sequences as well as hybrid RNA/DNA sequences of more than one nucleotide, indiscriminately in the simple chain form or in the duplex form.

The term “nucleotide” refers both to natural nucleotides (A, T, G, C) as well as to modified nucleotides comprising at least one modification such as (1) a purine analog, (2) a pyrimidine analog or (3) an analogous sugar, examples of such modified nucleotides being described for example in the PCT application WO 95/04 064.

As used in the present invention, a first polynucleotide is considered as being “complementary” to a second polynucleotide when each base of the first nucleotide is coupled to the complementary base of the second polynucleotide having the orientation inversed. The complementary bases are A and T (or A and U) or C and G.

The invention also relates to a nucleic acid comprising at least 20 consecutive nucleotides of the cDNA with a nucleotidic sequence SEQ ID N^{o} 2 coding for the plant glucosidase I according to the invention or a nucleic acid with a complementary sequence.

More particularly, the invention relates to a nucleic acid comprising the nucleic sequence SEQ ID N^{o} 2, or a nucleic acid with a complementary sequence.

Another object of the invention is a nucleic acid having at least 80% identity in nucleotides with one of the following nucleic acids:
a) a nucleic acid comprising at least 20 consecutive nucleotides of a polynucleotide coding for a glucosidase I having the aminoacid sequence SEQ ID N^{o} 1 or a nucleic acid with a complementary sequence;
b) a nucleic acid comprising at least 20 consecutive nucleotides having the nucleotidic sequence SEQ ID N^{o} 2 or a nucleic acid with a complementary sequence ; and
c) a nucleic acid comprising the nucleotidic sequence SEQ ID N^{o} 2 or a nucleic acid with a complementary sequence.

According to the invention, a first nucleic acid having at least 80% identity with a second reference nucleic acid will have at least 85%, preferably at least 90%, 95%, 98%, 99%, 99.5% or 99.8% identity in nucleotides with such a second reference polynucleotide, the identity percentage between two sequences being determined as described hereunder.

The “identity percentage” between two sequences of nucleotides or aminoacids, as meant in the present invention, can be determined comparing two optimally aligned sequences, through a comparison window.

The part of the nucleotide or polypeptide sequence in the comparison window can therefore comprise additions or deletions (for example “gaps”) compared to the reference sequence (which does not comprise such additions or deletions) so as to obtain an optimal alignment of both sequences.

The percentage is calculated through determining the number of positions where a nucleic base or an identical aminoacid residue is observed for both (nucleic or peptidic) sequences being compared, subsequently dividing the number of positions where there is an identity between both bases or aminoacid residues by the total position number in the comparison window, followed by multiplying the result by one hundred so as to obtain the sequence identity percentage.

The sequence optimal alignment for the comparison may be achieved by using a computer with known algorithms contained in the software of the company WISCONSIN GENETICS SOFTWARE PACKAGE, GENETICS COMPUTER GROUP (GCG), 575 Science Doctor, Madison, Wis.

Most preferably, the identity percentage between two sequences is computed using the BLAST software (BLAST 2.06 version, dated September 1998), using exclusively the default parameters (S. F. ALTSCHUL et al., 1990; S F ALTSCHUL et al., 1997).

Each of the nucleic acids according to the invention comprising all or part of the mRNA or cDNA corresponding to the transcription products of the AtGCS1 gene coding for a plant glucosidase I may be easily obtained by the man skilled in the art when knowing its nucleotidic sequence as disclosed in the present specification.

The man skilled in the art may also reproduce any one of nucleic acids according to the invention by creating, based on the sequences disclosed in the present specification, oligonucleotidic primers able to amplify all or part of such nucleic acids, for example, by extracting the total RNA from several plant tissues, then synthesizing the complementary DNA by means of an inverted transcriptase enzyme before performing several cDNA amplification cycles obtained by means of one or more primers specifically hybridizing with the target sequences the obtention of which is sought.

Such a reproduction mode of the nucleic acids according to the invention is described for instance in example 1.

After a specific amplification of a nucleic acid according to the invention through appropriate primers, the various amplified nucleic acids can then be subjected to a ligation step in a vector according to methods well known to the man skilled in the art.

Generally, a nucleic acid having at least 20 consecutive nucleotides with a sequence according to the invention has advantageously at least 25, 30, 35, 40, 50, 75, 100, 150, 200, 300, 400, 500, 1 000, 2 000 or 2 500 consecutive nucleotides of the reference sequence, the length of the consecutive nucleotides being naturally limited by the length of the reference sequence.

The man skilled in the art can thus reproduce a nucleic acid according to the invention through direct chemical synthesis, such as the phosphodiester method described by Narang et al. (1979), the phosphodiester method described by Brown et al. (1979), the diethylphosphoramidite method described by Beaucage et al. (1981), as well as the method on a solid support described in the European patent application n^{o} EP 0 707 792, each of which is incorporated herein by reference.

A nucleic acid according to the invention may also be synthesized from the information of reference polynucleotidic sequence having the SEQ ID N^{o}2 and SEQ ID N^{o} 5, by means of the methods described by Wheeler et al. (1996, Gene, Vol. 10:251-255), Wade et al. (1996, Biomedical Peptides, Proteins and nucleic acids, Vol. 2:27-32), Martinez et al. (1996, Taxicon, Vol. 34 (11) :1413-1419), Prapunwattana et al. (1996, Molecular and Biochemical Parasitoly, vol. 83 :93-106) and Skopek et al. (1996, Mutation Research, vol. 349 :163-172).

As already mentioned, the Applicant has also isolated and characterized, in an Arabidopsis thaliana ecotype, the Wassilevskja ecotype, a plant in the genome of which the AtGCS1 gene coding for the glucosidase I according to the invention has been artificially interrupted, by inserting a construction containing an Agrobacterium tumefaciens DNA-T.

After sequencing the AtGCS1 gene at the DNA-T level, it has been established that the exogenous DNA has been inserted at the beginning of the eighth intron. The insertion of the exogenous DNA has besides created a deletion of 30 pb of the genomic DNA as well as an insertion of two nucleotidic fragments with a respective length of 26 and 22 pb, on both sides of the DNA-T.

Accordingly, the Applicant has isolated and characterized a Atgcs1 gene comprising, compared to the naturally occurring AtGCS1 gene sequence, several nucleotide additions and deletions in the coding sequence.

It has been shown according to the invention that plants having a mutated Atgcs1 gene could more particularly be characterized through the phenotype of their seeds which, as far as one fourth of them are concerned, are wrinkled and unable to germinate.

It has further been shown that a mutation of the AtGCS1 gene, when present at the homozygote state in the genome, is lethal to the plant.

Moreover, it has been shown according to the invention that the glycosylation of the proteins expressed in plants within which the expression of the Atgcs1 is strongly altered, or even completely blocked or non existing, was deeply modified and that in particular the glycans present at the level of the glycosylation sites of such proteins were completely exempt of allergenic oligosaccharide residues such as fucose or xylose.

Accordingly, the genome sequence of the Atgcs1 gene as well as the nucleotidic sequence SEQ ID N^{o} 2 according to the invention are useful, more particularly, for implementing several means adapted to inhibit or block the synthesis of the glucosidase I coded by the AtGCS1 gene. The nucleotidic sequence SEQ ID N^{o} 2 according to the invention is also useful for implementing several means for specifically detecting the Atgcs1 gene or the transcription product thereof, such detecting means allowing the man skilled in the art to determine whether the interesting plant contains in its genome a functional AtGCS1 gene or, on the contrary, a mutated Atgcs1 gene, provided that the detection of the presence of at least one copy of the mutated Atgcs1 gene in the genome of a plant makes it possible to select such a plant in order to produce proteins non allergenic to man.

A nucleic acid such as defined hereunder codes for at least part of the glucosidase I according to the invention and may be inserted in particular in a recombinant vector adapted for the expression of the corresponding translation product in a host cell or in a plant transformed with such a recombinant vector.

Such a nucleic acid may also be used for the synthesis of nucleotidic probes and primers designed for detecting or amplifying the nucleotidic sequences included in the genomic DNA, the messenger RNA or also the cDNA of the AtGCS1 gene in a sample.

In order to perform the detection, nucleic acids can also be used with a complementary sequence to those defined hereunder.

Also included in the scope of the invention are the nucleic probes and primers hydridizing, in highly stringent hybridization conditions, with a nucleic acid with the nucleotidic sequence SEQ ID N^{o} 2.

It is understood by highly stringent hybridization conditions as used herein, the following hybridization conditions:
the DNA to be tested is immobilized on membranes of the GenScreenPlus®NEN™ Life Science Product type following the manufacturer instructions, in the presence of 0.4 M NaOH overnight;
the membranes are washed using a 2×SSC (1×SSC . . . idem) buffer, then are prehybridized at least 30 minutes at 65° C. in a hybridization buffer (Buffer: 7% SDS, 0.25 M Na₂HPO₄, pH 7.4, 2 mM EDTA, 20 mg/l heparin, 0.1 mg/l single strand DNA from calf thymus);
the probes are added to the membranes and incubated overnight at 65° C. ;
after the hybridization step, the membranes are washed in a 2×SSC buffer, 0.5% sarcosyl, 0.2% sodium pyrophosphate at 65° C. for 30 minutes ; and
a second wash is performed in a 0.2×SSC buffer, 0.5% sarcosyl, 0.2% sodium pyrophosphate at 65° C. for 10 minutes.

The above-mentioned hybridization conditions are adapted for hybridization under highly stringent hybridization conditions of a molecule of a nucleic acid with a length of 300 to 400 nucleotides.

It is obvious that the above-described hybridization conditions can be adapted depending on the length of the nucleic acid the hybridation of which is being sought or on the chosen marking type, according to techniques known to the man skilled in the art.

The appropriate hybridization conditions can, for example, been adapted according to the teaching found in the book by HAMES and HIGGINS (1985) or also in the book by AUSUBEL et al. (1989).

The nucleotidic probes or primers according to the invention comprise at least 15 consecutive nucleotides of a nucleic acid according to the invention, more particularly, a nucleic acid with a SEQ ID N^{o} 2 sequence or the complementary sequence thereof, a nucleic acid having at least 80% nucleotide identity with the SEQ ID N^{o} 2 sequence or the complementary sequence thereof or also a hybridizing nucleic acid, in highly stringent hybridization conditions, with the SEQ ID N^{o} 2 sequence or the complementary sequence thereof.

Preferably, nucleotidic probes or primers according to the invention have a length of at least 15, 20, 25, 30, 35, 40, 50, 75, 100, 150, 200, 300, 400 or 500 consecutive nucleotides of a nucleic acid according to the invention, in particular the nucleic acid with the SEQ ID N^{o} 2 nucleotidic sequence, or a nucleic acid with a complementary sequence.

According to another aspect, one nucleotidic probe or primer according to the invention will consist in and/or comprise fragments with a length of 15, 20, 25, 30, 35, 40, 50, 75, 100, 150, 200, 300, 400 or 500 consecutive nucleotides of a nucleic acid according to the invention, more particularly the nucleic acid with the SEQ ID N^{o} 2 sequence, or a nucleic acid with a complementary sequence.

Examples of primers or primer couples are for example SEQ ID N^{o} 3 and SEQ ID N^{o} 4 sequences, which make it possible to amplify the totality of the open reading frame of the messenger RNA of the Atgcs1 gene.

These may also be primers of SEQ ID N^{o} 5 to SEQ ID N^{o} 15 sequences according to the invention.

A nucleotidic primer or probe according to the invention may be prepared using any adapted method well known to the man skilled in the art, including cloning and action of restriction enzymes or even by direct chemical synthesis according to techniques such as the above-mentioned phosphodiester method of Narang et al. (1979) or Brown et al. (1979).

Each of the nucleic acids according to the invention as well as the oligonucleotidic probes and primers as described hereabove, can be marked, if desired, by incorporating a marker detectable through spectroscopic, photochemical, biochemical, immunochemical as well as chemical means.

For example, such markers can comprise radioactive isotopes (³²P, ³H, ³⁵S), fluorescent molecules (5-bromodeoxyuridin, fluorescein, acetylaminofluorene, digoxygenin) or also ligands such as biotin.

Marking a nucleic acid is done preferably by incorporation of marked molecules within such nucleotides through primer extension or also through addition on the 5′ or 3′ ends.

Examples of non radioactive marking of nucleic acid fragments are described in particular in the patent FR 78 19 175 or also in the publications by URDEA et al. (1988) or SANCHEZ-PESCADOR et al. (1988).

Advantageously, the probes according to the invention may have structural features the nature of which allows the signal to be amplified, such as the probes described by URDEA et al. (1991) or also in the European patent EP 0 225 807 (Chiron).

The oligonucleotidic probes according to the invention may be used in particular in hybridizations of the Southern type with genomic DNA or also in hybridizations with messenger RNA of the Atgcs1 gene, when a visualisation of the expression of the corresponding transcript is sought in a sample.

The probes according to the invention can also be used for detecting PCR amplification products or also for detecting mismatchings.

Nucleotidic probes or primers according to the invention can be immobilized on a solid support. Such solid supports are well known to the man skilled in the art and comprise surfaces of the microtitration plate wells, polystyrene balls, magnetic balls, nitrocellulose strips or also microparticles such as latex particles.

The present invention also relates to a method for detecting the presence of a nucleic acid of the AtGCS1 gene in a sample, said method comprising the steps of:
contacting a probe or a plurality of nucleotidic probes according to the invention with the sample to be tested adapted likely to contain a nucleic acid of the AtGCS1 gene ; and
detecting the hybrid potentially formed between the probe(s) and the nucleic acid present in the sample.

According to a particular embodiment of the detection method according to the invention, the oligonucleotidic probe(s) is or are immobilized on a support.

According to another aspect, the oligonucleotidic probes comprise a detectable marker.

The invention additionally relates to a set or a kit for detecting the presence of a nucleic acid of the AtGCS1 gene (gDNA, cDNA, MRNA) in a sample, said set comprising:
a) one or more nucleotidic probes, such as described hereabove; and
b) if needed, the reagents necessary to the hybridation reaction.

According to a first aspect, the detection set or kit is characterized in that the probe(s) is or are immobilized on a support.

According to a second aspect, the detection set or kit is characterized in that the oligonucleotide probes comprise a detectable marker.

According to a particular embodiment of the detection kit described hereabove, such a kit will comprise a plurality of oligonucleotidic probes according to the invention which could be used for detecting the interesting target sequences of the AtGCS1 gene or also for detecting mutations in the coding regions of the AtGCS1 gene, more particularly in the nucleic acid with the SEQ ID N^{o} 2 sequence or a nucleic acid with a complementary sequence.

The nucleotidic primers according to the invention can be used for amplifying any nucleotidic fragment (gDNA, cDNA, MRNA) of the AtGCS1 gene and more particularly all or part of a nucleic acid with SEQ ID N^{o} 2 sequence.

Another object of the invention relates to a method for amplifying a nucleic acid of the ATGCS1 gene and more particularly a nucleic acid with a SEQ ID N^{o} 2 sequence or a fragment thereof or also a nucleic acid with a complementary sequence to the latter, contained in a sample, said method comprising the steps of:
a) contacting the sample in which the target nucleic acid presence is suspected, with a couple of nucleotidic primers according to the invention;
b) performing at least one amplification cycle of the nucleic acid contained in the sample ; and
c) detecting optionally the amplified nucleic acid.

According to the amplification method hereabove, at least one amplification cycle is performed of the nucleic acid contained in the sample prior to the detection of the optionally amplified nucleic acid, preferably at least 10 and most preferably at least 20 amplification cycles.

In order to implement the above-mentioned amplification method, any of the above-described nucleotidic primers can be advantageously used having the hybridization position localized respectively on the 5′ side and on the 3′ side of the region of the target nucleic acid of AtGCS1 the amplification of which is being sought, in the presence of the reagents required for the amplification reaction.

The invention has moreover as a object a set or a kit for amplifying a nucleic acid of the AtGCS1 gene (gDNA, cDNA or mRNA) according to the invention, and more particularly all or part of a nucleic acid of a SEQ ID N^{o}2 sequence, said set or kit comprising:
a) one couple of nucleotidic primers according to the invention; and
b) if needed, the necessary reagents for the amplification reaction.

Such an amplification set or kit will advantageously comprise at least one pair of nucleotidic primers such as described hereabove the hybridization position of which is localized respectively on the 5′ side and the 3′ side of the target nucleic acid of the AtGCS1 gene the amplification of which is being sought.

According to a preferred embodiment, primers according to the invention comprise all or part of a polynucleotide selected among SEQ ID N^{o} 3 and SED ID N^{o} 4 nucleotidic sequences.

The invention also relates to methods designed to inhibit or block the expression of the AtGCS1 gene in the cells of plant tissues, with a view to producing glycosylated proteins by non allergenic glycans, by appropriate techniques well known to the man skilled in the art.

In order to inhibit or block the expression of the AtGCS1 gene in a plant, the man skilled in the art can more particularly use antisense polynucleotides or also co-suppression techniques.

Accordingly, the invention also relates to an antisense polynucleotide able to specifically target a determined region of the AtGCS1 gene and more particularly, a determined region of the SEQ ID N^{o} 2 nucleotidic sequence, able to inhibit or to block its transcription and/or its translation. Such a polynucleotide meets the general definition of probes and primers according to the invention.

According to a first aspect, an antisense polynucleotide according to the invention hybridizes with a sequence corresponding to a sequence localized in an region of the 5′ end of the messenger RNA of the AtGCS1 gene, and most preferably, in the vicinity of the translation initiation codon (ATG) of the ATGCS1 gene.

In order to build such an antisense polynucleotide, the man skilled in the art could advantageously refer to the cDNA sequence of the AtGCS1 gene referred to as the SEQ ID N^{o}2 nucleotidic sequence.

According to a second aspect, an antisense polynucleotide according to the invention comprise a sequence corresponding to one of the sequences localized at the level of the exon/intron junctions of the AtGCS1 gene and preferably, to the sequences corresponding to a ligation site, which can be determined according to techniques well known to the man skilled in the art, based on the description of the sequences of the invention, more particularly the SEQ ID N^{o} 5 and SEQ ID N^{o} 2 sequences.

According to a third aspect, an antisense polynucleotide according to the invention comprises the whole cDNA corresponding to the transcription product of the AtGCS1 gene. Most preferably, an antisense polynucleotide according to the invention comprises the SEQ ID N^{o} 2 nucleotidic sequence or consists in the SEQ ID N^{o} 2 sequence.

In order to synthesize the antisense polynucleotides such as defined hereabove, the man skilled in the art could refer to the positions of the various exons and introns of the AtGCS1 gene in the SEQ ID N^{o} 5 nucleotidic sequence.

Generally speaking, the antisense polynucleotides should have a length and a melting temperature sufficient to allow for the formation of an intracellular duplex hybrid with a sufficient stability for inhibiting the expression of the AtGCS1 mRNA.

Strategies for building antisense polynucleotides are described, among others, by GREEN et al. (1986) and IZANT and WEINTRAUB (1984), each of which is incorporated herein by reference.

Methods for building antisense polynucleotides are also described by ROSSI et al. (1991) as well as in the PCT applications WO 94/23 026, WO 95/04141, WO 92/18522 and in the European patent application EP 0 572 287, each of which is incorporated herein by reference.

Advantageously, an antisense polynucleotide according to the invention has a length of 15 to 4 000 nucleotides. An antisense polynucleotide of the invention preferably has a length of 15, 20, 25, 30, 35, 40, 45 or 50 to 75, 100, 200, 500, 1 000, 2 000, 3 000 or 4 000 nucleotides.

Amongst the antisense polynucleotides according the invention are preferred those having respectively a length of about 300 nucleotides or a length of about 4 000 nucleotides.

In order to inhibit or block the expression of the AtGCS1 gene, one can also use simultaneously a plurality of antisense nucleotides such as defined hereabove, each of the antisense polynucleotides hybridizing with a region distinct from the AtGCS1 gene or the messenger RNA thereof.

Other implementation methods of the antisense polynucletides are for example described by SCZAKIEL et al. (1995).

Another object of the invention is any method well known to the man skilled in the art allowing to create modifications, for example, one or more additions, deletions or substitutions of at least one nucleotide in the sequence of the AtGCS1 gene, such modifications resulting in either an inhibition or a blocking of the transcription of the AtGCS1 gene, or in a defect in the ligation of the pre-messenger RNA, or an inhibition or a blocking of the translation of the mature messenger RNA, in the glucosidase I according to the invention, or also in the production of a mutated glucosidase I having a reduced or no catalytic activity.

Appropriate mutagenesis techniques of the genomic sequence of the AtGCS1 gene are described for example by Hohn and Puchta (1999).

A plant having its genome modified as decribed hereabove is able to synthesize non allergenic and/or non immunogenic proteins with a modified glycosylation, in particular all the proteins naturally produced by the plant and designed for the human or animal food, such as those described by Zeng et al. (1997).

Additionally, such a plant affected in the expression of the catalytic activity of the glucosidase I according to the invention can be used in order to produce determined non allergenic and/or non immunogenic recombinant proteins adapted for use in man or animal.

Preferably, the plants being made deficient in the catalytic activity of the glucosidase I according to the invention are used with a view to producing non allergenic immunogenic proteins and antigenic proteins adapted for preparing vaccines for the immunization of man and animal. Any type of recombinant immunogenic or antigenic peptide or protein can thus be produced by a plant the AtGCS1 gene of which has been subjected to at least one addition, deletion or substitution of one or more consecutive nucleotides.

According to another aspect, the N-glycans having been subjected to a partial maturation process due to the absence or to a reduced rate of glucosidase I being catalytically active in the plants in which the AtGCS1 gene has been modified as described hereabove, and which essentially comprise the (Glc₃ Man₇ GlcNAc₂) structure can be used, after separation and purification, as vectors for compounds of therapeutic interest towards determined target cells in mammals and in particular in man. In fact, such partially modified glycans have a particular affinity for lectins specifically expressed at the membrane surface of some cell categories and have already made possible to address antiviral agents towards hepatocytes and macrophages (Murray et al., 1987).

According to another preferred embodiment according to the invention, an overexpression of the AtGCS1 gene or the transcription product thereof or also the glucosidase I protein according to the invention will be sought.

A strong expression of the AtGCS1 gene in a plant can be achieved either by the overexpression of the AtGCS1 gene or by the insertion of the multiple copies of a polynucleotide coding for the glucosidase I according to the invention in the plant, or also by a combination of both strategies.

For the insertion of multiple copies of a polynucleotide coding for the glucosidase I according to the invention into the genome of a plant, one will advantageously use a recombinant vector according to the invention.

The invention also relates to a recombinant vector comprising a nucleic acid according to the invention.

Advantageously, such a recombinant vector comprises a nucleic acid selected amongst the following nucleic acids:
a) a nucleic acid comprising at least 20 consecutive nucleotides of a polynucleotide coding for a glucosidase I having the SEQ ID N^{o} 1 aminoacid sequence or a nucleic acid with a complementary sequence;
b) a nucleic acid comprising at least 20 consecutive nucleotides of the SEQ ID N^{o} 2 nucleotidic sequence or a nucleic acid with a complementary sequence;
c) a nucleic acid comprising a sequence having at least 80% identity in nucleotides with the SEQ ID N^{o} 2 nucleotidic sequence, or a nucleic acid with a complementary sequence; and
d) an antisense polynucleotide or a homopurine or homopyrimidine polynucleotide, such as defined hereabove, useful for inhibiting the expression of the Atgcs1 gene.

As used herein, “vector” means a circular or linear DNA or RNA molecule which is indiscriminately in the form of a single strand or a double strand.

A recombinant vector according to the invention is indiscriminately a cloning vector, an expression vector or more specifically an insertion vector, a transformation vector or an integration vector.

It can be a vector from bacterial or viral origin.

According to a first embodiment, a recombinant vector according to the invention is used with the aim of amplifying the nucleic acid which is inserted therein after transformation or transfection of the desired cell host.

According to a second embodiment, it is an expression vector comprising, besides a nucleic acid coding for a polypeptide according to the invention, in particular the polypeptide with the SEQ ID N^{o} 1 aminoacid sequence, regulatory sequences allowing to direct the transcription and/or the translation thereof.

Moreover, the recombinant vectors according to the invention could include one or more replication origins in the cell hosts wherein their amplification or their expression is sought as well as selection markers.

In a particular embodiment, a recombinant vector according to the invention comprises an antisense polynucleotide or a homopurine or homopyridine polynucleotide, such as previously defined, optionally under the control of appropriate regulation sequences allowing to ensure its expression in a host cell or a selected plant. Such a recombinant vector is preferably used for inhibiting the Atgcs1 gene expression in the cell or in the plant.

According to another particular embodiment, a recombinant vector according to the invention comprises a polynucleotide coding for the ATGCS1 polypeptide or a polypeptide having at least 80% identity in aminoacids with the latter and maintaining the biological activity of ATGCS1, under the control of the regulation sequence(s) allowing for an expression at high level of ATGCS1 or the homolog thereof in a host cell or in a selected plant. Such a recombinant vector is useful for allowing for a high expression level of ATGCS1 in a plant.

According to an advantageous aspect, such a recombinant vector is an integrative vector allowing for the insertion of multiple copies of the ATGCS1 coding sequence into the genome of a plant.

As an example, the bacterial promoters could be Lacl, LacZ promoters, polymerase RNA promoters of the T3 or T7 bacteriophage, PR or PL promoters of the lambda phage.

Promoters for the expression of a nucleic acid coding for a glucosidase I according to the invention in plants are the 35S CaMV promoter of the cauliflower mosaic disease virus (Odell et al., 1985) or also the promoter of the gene of the actine 1 of rice (McElroy et al. 1990).

The FAH promoter can also be used (Patent FR 9907362) which allows to express a gene in a strong and constitutive way in all the plant parts except the seeds, useful for the sense and antisense strategies, as well as inducible promoters of the two component system (re McNellis T. W. et al. 1998, “Glucocorticoid-inducible expression of bacterial avirulence gene in transgenic arabidopsis induces hypersensitive cell death”. Plant Journal, 14 (2):247-257).

Other promoters useful for the expression of a polynucleotide of interest in plants are described in the U.S. Pat. Nos. 5,750,866 and 5,633,363, each of which is incorporated herein by reference.

Generally speaking, for the selection of the adapted promoter, the man skilled in the art could advantageously refer to the above-mentioned book by Sambrook et al. (1989) as well as to the techniques disclosed by Fuller et al. (1996) and Ausubel et al. (1989).

The preferred bacterial vectors according to the invention are for example pBR 322 vectors (ATCC N^{o} 37017) as well as vectors such as pAA223-3 (Pharmacia Uppsala, Sweden) and pGEM1, pBSSK and pGEM-T (Promega Biotech, Madison, WU, USA) and pUC19 (sold by Boehringer Mannheim, Germany).

Other commercialized vectors can be mentioned such as the pQE70, pQE60, pQE9 vectors (Qiagen, psuX 174, pBluescript SA, pNH8A, pMH16A, pMH18A, pMH46A, pWLNEO, pSG2CAT, pOG44, pXTI, pSG (Stratagene).

It can also be vectors of the baculovirus type such as the pVL1392/1393 vector (Pharmingen) used for transfecting cells of the Sf9 strain (ATC N⁰ CRL 1711) derived from Spodoptera Frugidera.

Preferably, one will use vectors specially adapted for the expression of the sequence of interest in the plant cells, such as the following vectors:
pBIN19 vector (Bevan et al., Nucleic Acids Research, Vol.12:8711-8721, commercialized by Clontech Corporation, Palo Alto, Calif., USA);
pB101 vector (Jefferson 1987, Plant Molecular Biology Reporter, vol. 5:387-405, commercialized by the Clontech Corporation);
pBI121 vector (Jefferson et al. 1987, Plant Molecular Biology Reporter, vol. 5:387 :405, commercialized by the Clotench Corporation);
pEGFP vector (Cormack BP et al., 1996, commercialized by the Clotench Corporation);
pAOV, pOV2, pSOV, pSOV2, pkMB and pSMB vectors (Mylne and al., 1996).

In order to allow the expression of the polynucleotides according to the invention, such vectors should be introduced in a host cell. The introduction of the polynucleotides according to the invention in a host cell could be achieved in vitro, according to techniques well known to the man skilled in the art for transforming or transfecting cells, either in a primary culture or in the form of cell strains.

Yet another object of the invention is a host cell transformed with a nucleic acid or by a recombinant vector according to the invention.

Such a transformed cell host is preferably from prokaryote or eukaryote origin, more particularly from bacterial, fungal or plant origin.

Accordingly, bacterial cells from various E. coli as well as Agrobacterium tumefaciens strains can be used among others.

Preferably, the transformed host cell is a plant cell or a plant protoplast.

Most preferably, it is a cell or a protoplast of rape, tobacco, corn, barley, wheat, alfalfa, tomato, potato, banana tree or Arabidopsis thaliana.

The invention also relates to a transformed plant multicellular organism, characterized in that it comprises a transformed host cell or a plurality of transformed host cells with a nucleic acid according to the invention or with a recombinant vector according to the invention.

According to a first aspect, the plant multicellular organism is transformed with one or more antisense nucleotides and/or one or more homopurine or homopyrimidine polynucleotides in order to inhibit or to block the expression of the AtGCS1 gene in that organism.

According to a second aspect, the plant multicellular organism is transformed with one or more copies of a polynucleotide coding for the glucosidase I according to the invention or for a polylpeptide with at least 80% identity in aminoacids with the glucosidase I and maintaining the biological activity allowing for the normal maturation of the glycans fixed on the glycosylation sites of the proteins being synthesized.

Yet another object of the invention is a transgenic plant, i.e., a transformed plant comprising, preferably in an integrated form into its genome, a nucleic acid of the Atgcs1 gene and preferably an antisense polynucleotide as well as a nucleic acid coding for the ATGCS1 polypeptide or a homologous polypeptide, said nucleic acid having been inserted into the plant genome by transformation with a nucleic acid of AtGCS1 or a recombinant vector according to the invention.

Preferably, a transformed plant according to the invention is a rape, tobacco, corn, soy, wheat, barley, alfalfa, tomato, potato, a fruit plant such a banana tree or Arabidopsis thaliana.

According to a first aspect, the transgenic plants such as defined hereabove show a reduced expression, an undetectable expression or a lack of expression of the AtGCS1 gene and are therefore able to allow for the reproduction of modified glycosylation proteins that are non allergenic and/or non immunogenic to man or to the animal. According to a particular embodiment, such plants synthetize a protein of interest having its coding sequence artificially introduced, such coding sequence being unable to be present under a non integrated form into the plant genome, or on the contrary, in an integrated form into the plant genome. The protein of interest may be of any nature, preferably a protein serving as feeding or therapy for man or animal, in particular immunotherapy or vaccination.

The invention also relates to a modified glycosylation protein, characterized in that it is produced by a transformed plant according to the invention, or also by a transformed host cell according to the invention, wherein the expression of the AtGCS1 gene is inhibited or blocked, or characterized in that it is contained in a seed of a transformed plant according to the invention. Preferably, the modified glycosylation protein is a recombinant protein. It can be a recombinant protein serving as feeding or therapy for human or animal. Such a recombinant protein can be an antigene or an immunogene useful in a vaccine composition.

The invention also relates to the addition of one more N-glycosylation site(s) in a recombinant protein of interest in order to modify the targeting and/or the stability thereof, in plant cells, a plant tissue or a plant transformed according to the invention.

According to a second aspect, the transgenic plants such as defined hereabove have the propriety to strongly express a glucosidase I according to the invention.

An additional object of the invention is a method for obtaining a transgenic plant transformed with a nucleic acid according to the invention, characterized in that it comprises the following steps of:
a) obtaining a plant transformed host cell as defined hereabove;
b) regenerating a whole plant from the transformed plant host cell obtained in step a) ; and
c) selecting plants obtained in step b), having integrated the nucleic acid of interest.

The invention also relates to a method for obtaining a transgenic plant transformed with a nucleic acid according to the invention, characterized in that it comprises the following steps of:
a) transforming a plant cell with a nucleic acid of the AtGCS1 gene or with a recombinant vector according to the invention;
b) regenerating a whole plant from the transformed plant cells obtained in step a) ; and

c) selecting plants having integrated the nucleic acid of the AtGCS1 gene of interest.

The invention also relates to a method for obtaining a transformed plant, characterized in that it comprises the following steps of:
a) obtaining a Agrobacterium tumefaciens host cell transformed with a nucleic acid or a recombinant vector according to the invention;
b) transforming the selected plant by infection with Agrobacterium tumefaciens cells obtained in step a) ; and
c) selecting plants having integrated the nucleic acid according to the invention.

Any method for obtaining a transgenic plant, as described hereabove, can additionally comprises the following additional steps of:
d) crossing with each other two transformed plants as obtained in step c); and
e) selecting heterozygous plants for transgene.

According to another aspect, any of the above-described methods could moreover comprise the following steps of:
d) crossing of a transformed plant as obtained in step c) with a plant of the same species ; and
e) selecting plants originating from the crossing in step d) having kept the transgene.

The man skilled in the art is able to implement numerous methods of the prior art with a view to obtaining plants transformed with a nucleic acid of the AtGCS1 gene according to the invention.

The man skilled in the art will advantageously refer to the technique as disclosed by BECHTOLD et al. (1993) in order to transform a plant through the Agrobacterium tumefaciens bacterium.

The techniques used in other vector types could also be used such as the techniques disclosed by BOUCHEZ et al. (1993) or also by HORSCH et al. (1984).

The man skilled in the art could also refer to the technique disclosed by Gomord et al. (1998).

Another object of the invention is a transformed plant such as obtained according to any of above-described obtaining methods.

The invention also relates to a plant seed having part or all the constitutive cells comprising a nucleic acid of the AtGCS1 gene according to the invention which has been artificially inserted in their genome.

Another further object of the invention is a transgenic plant seed such as defined hereabove.

The invention also relates to a plant cell comprising a nucleic acid of the AtGCS1 gene. Preferably, the nucleic acid of the AtGCS1 gene has a form integrated into the genome of said plant cell.

The invention also relates to a plant tissue made of a set of transformed plant cells such as hereabove defined.

Another object of the invention is to use a nucleic acid of the AtGCSt gene according to the invention for the expression in vitro or in vivo, preferably in plants, of the glucosidase I according to the invention or of a peptide fragment thereof.

The invention also relates to the use of an antisense nucleic acid according to the invention for inhibiting or for blocking the expression of the gene coding for the glucosidase I according to the invention.

Preferably, the above-mentioned uses are characterized in that it is an in vivo expression in a plant transformed with such a nucleic acid.

The invention also relates to using an antisense nucleic acid according to the invention for inhibiting or for blocking the in vivo or in vivo expression of the gene coding for the glucosidase I according to the invention and more particularly the glucosidase I with the SEQ ID N^{o} 1 aminoacid sequence.

The antisense nucleic acid or the homopurine or homopyrimidine nucleic acid is preferably used for inhibiting or blocking the expression of the in vivo expression of the AtGCS1 gene, in the transformed plant with such a nucleic acid.

The glucosidase I according to the invention with the SEQ ID N^{o} 1 aminoacid sequence has a length of 852 aminoacids. Such a protein has a calculated molecular weight of 97.5 kDa. After analysis of the sequence, the Applicant has identified a hydrophilic region containing several arginines having their polypeptide N-terminal part with a SEQ ID N⁰ 1 sequence, such a hydrophilic region containing the retention consensus signal in the endoplasmic reticulum of the type II membrane proteins, having the C-terminal end located in the lumen.

Such a hydrophilic region consists in the region ranging from the aminoacid in position I to the aminoacid in position 38 of the SEQ ID N^{o} 1 aminoacid sequence.

The glucosidase I according to the invention also comprises a hydrophobic region corresponding to a transmembrane domain, such hydrophobic region ranging from the aminoacid in position 38 to the aminoacid in position 17 of the SEQ ID N^{o} 1 aminoacid sequence.

The glucosidase I according to the invention comprises a single fixation site to glycan ranging from the aminoacid in position 598 to the aminoacid in position 606 in the SEQ ID N^{o} 1 aminoacid sequence. A single glycosylation site has also been identified, which ranges from the aminoacid in position 662 to the aminoacid in position 664 of the SEQ ID N^{o} 1 aminoacid sequence.

Moreover, the glucosidase I according to the invention comprises a large hydrophilic region, probably located in the lumen of the endoplasmic reticulum, extending from the aminoacid in position 68 to the C-terminal aminoacid in position 852 of the SEQ ID N^{o} 1 aminoacid sequence.

As already stated, the Applicant has shown that mutations in the sequence of the AtGCS1 gene coding for the glucosidase I of the SEQ ID N^{o} 1 sequence according to the invention leads to the lack of glucosidase I activity in the thus mutated plant and simultaneously to the expression of a modified glycosylation phenotype in the proteins produced by such a mutated plant.

According to another aspect, the invention also relates to a polypeptide coded by a nucleic acid of the AtGCS1 gene and preferably a polypeptide comprising at least 7 consecutive aminoacids of the glucosidase I with a SEQ ID N^{o} 1 aminoacid sequence.

Preferably, such a polypeptide comprises at least 20, 25, 30, 40, 50, 60, 70, 80, 90, 100, 150, 200, 250, 300, 350, 400, 450, 500, 600, 700 or 800 consecutive aminoacids of the ATGCS1 polypeptide with a SEQ ID N^{o} 1 aminoacid sequence.

The invention also relates to a polypeptide comprising aminoacid sequences having at least 80% identity in aminoacids with the SEQ ID N^{o} 1 sequence of the ATGCS1 polypeptide or to a peptidic fragment thereof.

Advantageously, the invention includes also a polypeptide having at least 60%, 80%, 85%, 90%, 95% or 99% identity in aminoacids with the sequence of the ATGCS1 polypeptide with the SEQ ID N^{o} 1 sequence, or a peptidic fragment thereof.

Generally speaking, the polypeptides according to the invention have an isolated or a purified form.

The invention also relates to a method for producing the ATCGS1 polypeptide with the SEQ ID N^{o} 1 sequence, or a peptidic fragment thereof.

A preferred method for producing ATGCS1 polypeptide with a SEQ ID N^{o} 1 sequence comprises the following steps of:
a) inserting a nucleic acid coding for the ATGCS1 polypeptide or a peptidic fragment thereof, in an appropriate vector;
b) cultivating, in an appropriate culture medium, a host cell preliminarily transformed or transfected with the recombinant vector in step a);
c) recovering from the conditioned or lysed culture medium the transformed host cell, for example by sonication or osmotic shock;
d) separating and purifying from the culture medium or also from the cell lysates obtained in step c), said polypeptide ; and
e) optionally, characterizing the resulting recombinant polypeptide.

The peptides according to the invention can be characterized by fixation on an immunoaffinity chromatography column on which the antibodies directed against such polypeptides or a fragment or a variant thereof have been previously immobilized.

According to another aspect, a recombinant polypeptide according to the invention can be purified by using on a chromatography column according to methods known to the man skilled in the art and disclosed for example by the above-mentioned AUSUBEL F. et al. (1989).

A polypeptide according to the invention can also be prepared using conventional chemical synthesis techniques indiscriminately in homogeneous solution or in solid phase.

As an example, a polypeptide according to the invention could also be prepared by the technique in homogeneous solution as disclosed in HOUBEN WEYL (1974) or also by the solid phase synthesis technique as disclosed by MERRIFIELD (1965a, 1965b).

Included in the invention are also the polypeptides co-called “homologous” to the ATGCS1 polypeptides or the fragments thereof.

Such homologous polypeptides have aminoacid sequences having one or more substitutions of an aminoacid by an equivalent aminoacid, compared to the reference polypeptide.

As used herein, equivalent aminoacids mean, for example, the replacement of a residue in the L form by a residue in the D form or also the replacement of a glutamic acid (E) by a pyroglutamic acid according to techniques well known to the man skilled in the art.

As an example, the synthesis of polypeptides containing at least one residue in the D form is described by KOCH et al. (1977).

According to another aspect, are also considered as equivalent aminoacids two aminoacids belonging to the same class, i.e. two acidic, basic, non polar or polar non loaded aminoacids.

Is also included in the invention a polypeptide comprising aminoacid modifications of 1, 2, 3, 4, 5, 10 to 20 substitutions, additions, deletions of an aminoacid compared to the aminoacid sequence of the ATGCS1 polypeptide according to the invention.

Preferably, the polypeptides according to the invention comprising one or more additions, deletions or substitutions of at least one aminoacid maintain their ability to catalyze the cleavage of the first glucose residue of the precursor N-glycan with a (Glc₃ Man₉ GlcNAc₂) structure which can easily determined by the man skilled in the art, for example by using techniques disclosed by Sambrook et al. (1989).

According to another preferred embodiment, the polypeptides according to the invention comprising one or more additions, deletions, substitutions of a least one aminoacid maintain their ability to be recognized by antibodies directed against the ATGCS1 polypeptide with the SEQ ID N^{o} 1 sequence.

The invention also relates to a nucleic acid coding for a polypeptide such as defined hereabove.

A polypeptide derived from the ATGCS1 protein is useful, especially for preparing antibodies adapted for detecting the presence of such a polypeptide or also a peptidic fragment thereof in a sample.

Beside detecting the presence of the ATGCS1 polypeptide or also a peptidic fragment thereof in a sample, antibodies directed against such polypeptides are used for quantifying the synthesis of glucosidase I, for example in the cells of a plant and for determining thereby the ability of such a plant to synthesize mature glycans at the level of the glycosylation sites produced by the cells of said plant.

Preferred antibodies according to the invention are antibodies specifically recognizing the aminoacid sequence ranging from the aminoacid in position 1 to the aminoacid in position 38 (N-terminal hydrophilic region) of the sequence of the ATGCS1 polypeptide of a sequence SEQ ID N^{o} 1.

A second class of preferred antibodies according to the invention are antibodies specifically recognizing the aminoacid sequence ranging from the aminoacid in position 39 to the aminoacid in position 67 (transmembrane domain) of the sequence of the ATGCS1 polypeptide of a sequence SEQ ID N^{o} 1.

A third class of preferred antibodies according to the invention are antibodies specifically recognizing the aminoacid sequence ranging from the aminoacid in position 68 to the aminoacid in position 852 (hydrophilic region comprising the catalytic site) of the ATGCS1 polypeptide of a sequence SEQ ID N^{o} 1.

A fourth class of preferred antibodies according to the invention are antibodies specifically recognizing the fixation site to the glycan of the ATGCS1 protein and, more particularly, those recognizing a peptide of a “ERHVDLRCW” sequence.

As used in the present invention, “antibody” means more particularly polyclonal or monoclonal antibodies or fragments (for example, F(ab)′₂, F(ab))fragments or also any polypeptide comprising a domain of the initial antibody recognizing the target polypeptide or polypeptide fragment according to the invention.

The monoclonal antibodies can be prepared from hybridomas according to the technique disclosed by KOHLER and MILSTEIN (1975).

The present invention also relates to antibodies directed against a polypeptide such as described hereabove or a fragment or a variant thereof, such as produced in the trioma technique or in the hybridoma technique disclosed by KOZBOR et al. (1983).

The invention also relates to single chain (Fv) antibody fragments (ScFv) such as disclosed in the U.S. Pat. No. 4,946,768 or by MARTINEAU et al. (1998).

The antibodies according to the invention also comprise antibody fragments obtained using phage banks (RIDDER et al. 1995, REINMANN K. A. et al., 1997).

The antibody preparations according to the invention are useful in immunological detection tests for identifying the presence and/or the amount of the glucosidase I according to the invention or a peptidic fragment of such a protein, being present in a sample.

An antibody according to the invention could additionally comprise an isotopic or non isotopic detectable marker, for example fluorescent, or be coupled to a molecule such as biotin, according to techniques well known to the man skilled in the art.

Accordingly, another further object of the invention is a method for detecting the presence of a plant glucosidase I or also a peptidic fragment of one of such polypeptides according to the invention in a sample, said method comprising the steps of:
a) contacting the sample to be tested with an antibody such as defined hereabove ; and
b) detecting the antigene/antibody complex being formed.

The invention also relates to a diagnosis set or kit for detecting the presence of a polypeptide according to the invention in a sample, said set comprising:
a) one antibody as described hereunder; and
b) optionally, a reagent necessary to the detection of the formed antigene/antibody complex.

The invention is further illustrated, without being in any way limited, by the following examples and figures.

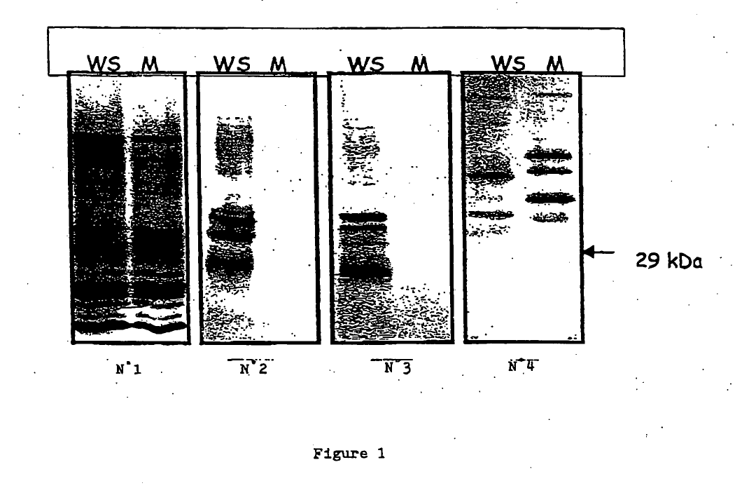

FIG. 1 shows electrophoresis gels of proteins extracted from cells of the Arabidopsis thaliana seeds of the wild type (Ws wild ecotype) and from cells of the ecotype seeds wherein the AtGCS1 gene has been interrupted by the insertion of a sequence of the DNA-T from Agrobacterium tumefaciens (M), also originating from the Ws ecotype.

Left hand-side N^{o} 1 gel is a SDS-PAGE electrophoresis gel on which the proteins which have migrated have been colored using silver nitrate.

N^{o} 2 gel has been incubated in the presence of an anti-xylose antibody.

N^{o} 3 gel has been incubated in the presence of an anti-fucose antibody.

N^{o} 4 gel has been incubated in the presence of concanavalin A.

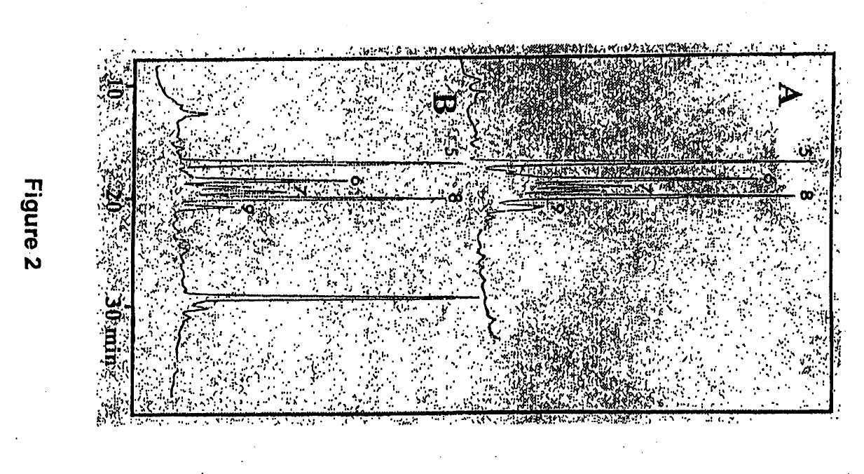

FIG. 2 illustrates HPAEC-PAD chromatograms of the N-glycans released from wild seeds (A) and the atgcs1 mutant (B) by Endo H treatment. The reference numerals 5 to 9 refer to the oligomannosidic N-glycan structures Man₅GlcNAc to Man₉GlcNAc represented in table 1.

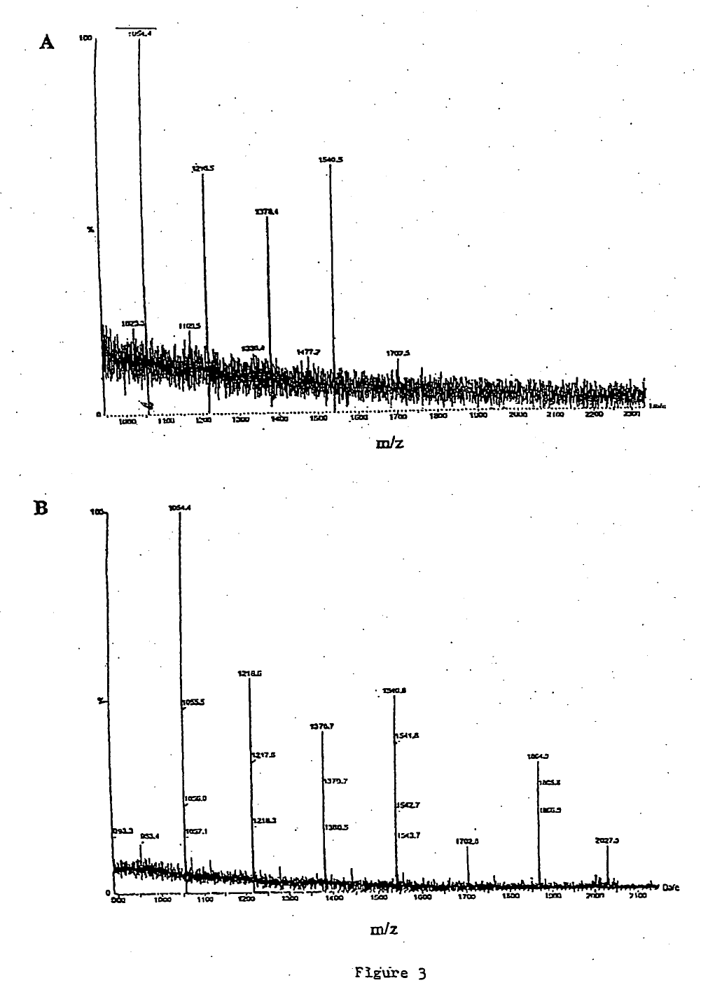

FIG. 3 illustrates MALDI-TOF mass spectra of N-glycans isolated from wild seeds (FIG. 3A) and seeds carrying the mutated AtGCS1 gene (FIG. 3B). In the abscissas, the m/z value represents the mass/load ratio.

### EXAMPLE 1

Isolation of cDNA Coding for Glucosidase I from Arabidopsis thaliana

1. RNA Extraction from Different Plant Tissues

The total RNA of 8 days-old young plants are extracted using the RNeasy Plant Minikit kit from QIAGEN (Germany) according to the protocol supplied by the manufacturer. A DNA degradation step has been systematically achieved on the extraction column (RNase free DNase, QIAGEN, Germany) according to the manufacturer's instructions. All the handlings are performed with a RNase free material and DEPC water is used (0.1% diethyl pyrocarbonate being autoclaved after a 12 hour stirring).

In vitro cultivated 8 days-old young plants are taken and placed immediately in liquid nitrogen and stored at −80° C. until extraction. Pipettes and material used for extracting RNA are previously treated with 0.4 M soda for destructing the possible RNases.

Water is DEPC treated (1 ml DEPC in 1 l water, homogeneization overnight under a Sorbonne), then autoclaved for withdrawing said agent. The column plus collecting tube centrifugations are carried out in “Micro Centaure” type centrifuges (MSE, UK).

The plant material is crushed in liquid nitrogen, in sterile Eppendorfs using an electrical grinder. The crushed material is immediately dipped in liquid nitrogen after grinding. The extraction as such is done with the “RNAeasy Plant Mini Extraction kit” kit (QIAGEN, Germany). Briefly stated, 450 μl “RLT” lyse buffer, added with β-mercaptoethanol (10 μl/1 ml), are vortexed with 100 mg ground tissue.

The next steps are exactly similar to those described in the kit instruction manual supplied by QIAGEN. The lysate, placed in a column and its 2 ml collecting tube, is centrifuged for 2 minutes.

The filtrate is recovered and washed with 0.5 volume of 96-100% ethanol. The sample is then placed on a column and its 2 ml collecting tube in order to be centrifuged for 15 seconds at 10,000 rpm, the RNA are retained by the column. 350 μl of RW1 washing buffer are added on that column in order to wash the RNA with a 15 second centrifugation at 10,000 rpm. A DNase treatment is then performed (10 μl DNase and 70 μl buffer, QIAGEN) for 15 minutes at room temperature. RW1 (350 μl) washing buffer is again applied to complete the washing cycle. The column is then placed in a new 2 ml collecting tube and two successive washing cycles are performed in 500 μm pf the RPE washing buffer added with ethanol. Finally, the RNA are eluted from the column by 30 to 50 μl DEPC treated water after a 1 minute centrifugation at 10,000 rpm. The RNA are stored at −80° C.

2. Reverse PCR (Reverse Transcriptase-PCR)

The single strand cDNA, as well as the amplification products are obtained with the “Enhanced avian RT-PCR kit” system (SIGMA, USA).

The retrotranscription occurs under the following conditions:
10 μl of the RNA extraction
1 μl of a deoxynucleotide mixture
1 μl dT oligonucleotide
4.5 μl H₂O (qsp 16.5 μI) for 10 minutes at 70° C. in order to denature the RNA, and the following materials are added to it:
2 μl 10×AMV-RT buffer
1 μl of the Enhancer avian RT enzyme
0.5 μl of RNase inhibitor.

The mixture is incubated for 15 minutes at 25° C. in order to allow the hybridation of the dT oligonucleotide, and then the retrotranscription occurs for 50 minutes at 42° C. The synthesized DNAc are then amplified by PCR.

The PCR reaction has been conducted on a thermocycler (MJ Research PTC 100-96, Prolabo, France), in 0.2 ml tubes (Prolabo, France) containing the following mixture:
2 μl/20 μl of the RT product
5U enzyme Pfu Turbo™ DNA Polymerase (Stratagene, USA)
10 μl Pfu 10× buffer
4 μl 5mM dNTP (GIBCO BRL, Scotland)
2 μl of each 10 μM oligonucleotide (GENSET, France) qsp 100 μl H₂O

Oligonucleotides specific to the AtGCS1 have been selected to amplify the coding sequence. These are “ATG” (5′ ATGACCGGAGCTAGCCGTCG) and “FO” (5′ AAGTTTCGTTCCCGAAGAGG), respectively located on the putative ATG and at 30 pb downstream of the T1F15.4 putative stop.

The reaction has been conducted under the following conditions:

1 initial denaturation step at 94° C. for 3 minutes, followed by 35 cycles, each cycle comprising the following steps:
94° C. : 30 sec
60° C. : 1 min
72° C. : 3 min
an elongation step at 72° C. for 10 minutes.

### EXAMPLE 2

Identification of the AtGCS1 Gene

The identification of the atgcs1 interrupted gene has been performed by isolating the DNA-T genomic edges originating from the A. thalmiana atgcs1 ecotype (insertion mutant) by the “PRC running” technique, (walk-PCT) described by Devic et al. (1997).

The sequences of the thus cloned DNA fragments have been compared to those obtained in the databases. Such sequences show a very high similarity to the bac T1F15 sequence originating from the TAMU Arabidopsis thaliana DNA and recorded in the GenBank database under the reference AC004393.

The Arabidopsis thaliana DNA insert contained in the bac T1F15 is a portion of the 1 chromosome and has been mapped as being located between the cM98 and cM99 positions of the 1 chromosome, i.e. on an region of the 1 chromosome at the opposite end of the centromere.

A second rather high similarity of 66% identity in nucleotides has also been identified with the Arabidopsis thaliana DNA insert contained in the bac F316 of the IGF bank. The insert of the bac F316 has also been mapped on the 1 chromosome between the cM30 and cM40 positions.

The intron/exon exact structure of the Atgcs1 gene has been established by aligning the cDNA sequence with that of the genomic clone recorded in the GenBank database under the reference AC004393. Differences are to be noted compared to the above-mentioned coding sequence of T1F15.4 gene in the databases. The Atgcs1 gene actually exhibits 22 exons and 21 introns. The exons N^{o} 4 and 16 have been added and the exons n^{o} 5, 6, 15 and 17 shortened compared to the base predictions. The AG ligating site at the intron end and the GT ligating site at the beginning are retrieved each time, except for the ninth intron which starts with GC. There are also some punctual differences. They can be due to the difference in the ecotypes being used for making the BAC bank (Columbia) and/or for amplifying the cDNA (Wassilevskija).

### EXAMPLE 3

Analysis of the Structure of the Arabidopsis thaliana Glucosidase I Coded by the AtGCS1 Gene

1. Sequence Similarity with Other Real or Putative Glucosidases

The AtGCS1 protein has a size of 852 aminoacids and a molecular mass estimated to be 97.5 kDa. This indeed corresponds to the bibliography data, the glucosidases I purified so far having a molecular mass ranging from 85 to 95 kDa (Pukazhenthi et al., 1993). From this new protein sequence, the research on the databases has been enhanced. The table hereunder shows the similarity between the AtGCS1 proteins and the already characterized glucosidases I.

| | Identified AtGCS1 | AtGCS1 similarity |
|---|---|---|
| Man (shown activity) | 38 | 54 |
| Mouse (cloned gene) | 38 | 55 |
| | 38 | 55 |
| (putative) C. elegans | 36 | 53 |
| (putative) S. pombe | 31 | 45 |

The identity (38%) and similarity (54%) percentages obtained between the AtGCS1 sequence and the human protein are insufficient to conclude to the glucosidase I activity of the AtGCS1. The identity and similarity percentages have been obtained using the DNA STRIDER™ 1.3 software.

2. Comparison with Peptide Fragments of a Purified Plant Glucosidase I

The glucosidase I of young bean plants (Vigna radiata, Zeng and Albein, 1998) has been purified and the sequencing of four peptides has been performed. The sequences of these four peptides are aligned with the protein sequence of ATGCS1 and glucosidase I in man.

| | | | |
|---|---|---|---|
| Peptide 1 | NYQQSGFLWEQYDQIK | Peptide 3 | DFG.QVLVDIGM |
| ATGCS1 810 | NYYETGYIWEQYDQVK | ATGCS1 168 | DYGRQELVENDM |
| Man 800 | QYQATGFLWEQYSDRD | Man 155 | SFGRQHIQDGAL |
| Peptide 2 | SLLWTNYGLR | Peptide 4 | EDIGDWQLRFK |
| ATGCS1 741 | SILWSDYGLV | ATGCS1 253 | EDVGDWQIHLK |
| Man 719 | RHLWSPFGLR | Man 178 | QHGGDWSWRVT |

The 1 peptide identified in bean makes it possible to retrieve in the databanks the homologous sequence in Arabidopsis. However, such data alone are not sufficient to show that the thus identified gene (T1F15.4) codes for the functional homolog of the bean Glucosidase I. Such a data is of the same level as the indications from the databases regarding T1F15.4 (putative gene) which, moreover, do not allow to obtain the true coding sequence.

3. Retention Pattern in the Endoplasmic Reticulum

The glucosidase I is a membrane protein of the II type (the C-terminal end is located on the lumen) and should have, as the proteins located in the lumen, a retention consensus signal in the RE of the type: two arginines in the first AA in N-term (Shütze et al., 1994). The N-terminal protein sequence (MTGASRR) has two arginines in the first aminoacids (for man, the sequence starts with: MARGER).

4. Hydrophobicity Profile

A hydrophilic region extends from residues 1 to 38, which could correspond to the cytoplasmic domain of the human protein (1 to 39). Subsequently, a hydrophobic domain extends as far as the aa 67, which corresponds to the transmembrane domain of the human protein (39 to 5). The remainder of the protein is rather hydrophilic, this is the part probably located in the lumen.

5. N-glycosylation Site

The glucosidases I characterized at the biochemical level all have a single N-glycosylation site, which is to be found in an equivalent position in the human protein (NHT 657-659) and in the Arabidopsis protein (NHT 662-664).

6. Fixation Site to the Substrate

The fixation site to the glycan has been described in the human protein as being the ERHLDLRCW peptide (Romaniouk et Vijay, 1997). Such a peptide is located in position 594. In the ATGCS1 protein, a similar peptide (ERHVDLRCW) is to be found in position 598.

### EXAMPLE 4

Cytologic Analysis on the Tissues and the Seeds of Arabidopsis Thaliana Plants having their AtGCS1 Gene Interrupted by the Agrobacterium Tumefaciens DNA-T

Preliminary observations have shown that, up to the embryo's core stage, no phenotype differences can be observed between the seeds. Thereafter, the wild and heterozygous embryos (which are identical) differ from one another and proceed with their development, whereas the homozygous plant for mutation on the Atgcs1 gene remains at the core stage and increases in volume. The mutated plant by no means differs, the hypocotyle thereof is quite reduced, the cotyledons and the root thereof are not elongated. The sections made during the embryo development in the mutant plant show that the albumen forms normally cells. The cells seem bigger and less numerous than those of the wild plant with an identical age, mainly at the level of the most external layer, the protoderm. In the mature homozygous embryo, vascular bundles in the reduced hypocotyle and a small apical meristeme can be distinguished. The epiderm is highly altered, the cells are enormous and shows “empty” spaces. The radicle seems very little developed, it is by no means differentiated.

An approach in transmission electronic microscopy has made it possible to better visualize the cell disorganization of the homozygous mutant. The observations have been made in the cotyledons and in the protoderm. The protein bodies, in the non mutated wild plant, have organized circular structures and shows little dense regions. 2 to 5 such regions can be visualized per cell. The lipid bodies fill the whole cell, a core being observed in each cell. In the mutant plant, the lipid bodies are not altered, they are present in a large number and fill nearly the whole cell. The protein bodies, on the other hand, are altered. They are not present in all the cells and, when one can be observed, it is not circular and generally surrounds a vacuole. In each cell are located one or more vacuoles, with a more or less dense content, which is never observed in the wild non mutated plant.

### EXAMPLE 5

Biochemical Analysis: Evidence of the Absence of Glucosidase I Activity in the Seeds of the Mutated Plant on the Atgcs1 Gene

5.1 Materials and Methods

The methods used are described in Fichette-Laine et al., (1998) Methods in Biotechnology 3 :271-290.

Protein Electrophoresis

The extraction and the transfer of the proteins of the electrophoresis gel on a nitrocellulose membrane (Schleicher & Schuell, BA 85, 0.45 μm) occur following the technique described by Towbin et al. (1979, Proc. Natl. Acad. Sci. USA, 76:4350-4354). During the electrophoresis, the material used for the transfer (nitrocellulose membrane, 3 MM Whatmann paper, scotch brite) is equilibrated 15 to 30 minutes in the transfer buffer (25 mM Tris; 20 mM glycin; methanol 10%). At the end of the electrophoresis, the transfer cassette is placed in a transfer vessel containing 600 ml of the above-described buffer. A satisfactory transfer of the electrophoresis gel proteins on the nitrocellulose membrane is achieved in 2 hours under a 10 V.cm⁻¹ electrophoresis field. An efficiency check of the transfer is achieved by a reversible coloration of the nitrocellulose membrane with Ponceau S red (1% (w/v) in 3% TCA). The discoloration of the nitrocellulose membrane after Ponceau S red treatment is achieved by rinsing with a TBS buffer (500 mM NaCl; 20 mM Tris-HCl, pH 7.4). All these treatment steps of the nitrocellulose membrane, so-called fingerprint after the transfer, are conducted under mild stirring at room temperature.

Immunodetection

Once the membrane equilibrated in the TBS, the nitrocellulose coupling sites still available after the protein transfer are saturated by a one hour incubation in a saturation solution (3% gelatin dissolved in the TBS buffer). After saturation of the membrane, the immunodetection of the proteins is achieved. The fingerprint is incubated for 90 minutes in the presence of a 1/1 000^{th} diluted rabbit polyclonal immunizing serum in a 1% gelatin solution (w/v) in the TBS buffer. After this incubation, the non fixed antibodies are eliminated by a series of four 15 minute washing cycles in TTBS buffer (TBS buffer+0.1% Tween 20). The fingerprint is then subjected to an incubation in the presence of a second antibody coupled to the horseradish peroxydase (goat IgG, rabbit antilgG, coupled to the horseradish peroxydase, Bio-Rad). This second antibody is 1/3 000^{th} diluted in a 1% gelatin solution (w/v) in the TBS buffer for 90 minutes. The conjugated second antibody excess is eliminated by four 15 washing cycles in TTBS buffer. The Tween 20 is eliminated at the end of the treatment by a 15 minute washing cycle of the fingerprint in the TBS buffer. The proteins recognized by the rabbit IgG immunoglobulines are evidenced by incubation of the membrane in a mixture containing 30 mg 4-chloro-1-naphthol (HRP color reagent, Bio-Rad) dissolved in 10 ml methanol and added with 50 ml TBS buffer containing 30 μl 30% H₂O₂.

Affinodetection of Proteins on a Membrane by Concanavalin A

The concanavalin A (ConA) is a lectine of a Canavalia ensifonnis leguminous plant which specifically recognizes the β-linked mannose residues of the oligomannosidic glycans associated to the proteins and also to the peroxydase. Once the membrane equilibrated in the TBS, the nitrocellulose coupling sites still available after the transfer are saturated by a one hour minimum incubation in TTBS buffer. The membrane is then incubated for 90 minutes in TTBS buffer added with salts (1 mM CaCl₂; 1 mM MgCl₂) allowing for the activation of the lectine and containing 25 μg.mL⁻¹ ConA (Sigma). After this incubation, the membrane is washed in four 15 minute baths in salt complemented TTBS in order to eliminate the ConA being not fixed or fixed on non specific sites. The membrane is then incubated for 60 minutes in TTBS complemented with salts in the presence of 50 μg.mL⁻¹ horseradish peroxydase.

The peroxydase excess is eliminated by four successive 15 minute washing cycles in TTBS complemented with salts. A washing cycle of the fingerprint in TBS buffer added with salts allows for the elimination of Tween 20. The evidence of the peroxydase activity is made available as described for the immunodetection.

5.2 Results

The analysis of the N-glycosylation of the proteins of homozygous seeds has been achieved and the results obtained are given in FIG. 1.

The detection of the precursors, the N-glycans, has been achieved by concanavalin A; this is a lectine specifically linking to the non mature glycans. Numerous additional strips appear in the mutant, there is therefore a high precursor accumulation. The detection of the complex oligosaccharides is achieved by means of anti-xylose and anti-fucose antibodies; all the strips disappear in the mutant, there is no complex N-glycan. The mutated plant is therefore indeed affected in the N-glycan maturation at the level of the glucosidase I.

The above-mentioned results show well that the Atgcs1 mutant is unable to catalyze the addition reaction of a xylose residue in a β1-2 position or a fucose residue in an α1-3 position on the glycans of the glycoproteins it produces.

Moreover, the profile of the glycoproteins exhibiting glycans of the oligomannosidic type (fingerprint 4) is strongly modified in the mutant.

### EXAMPLE 6

Analysis of the Glycans Fixed on the Synthetized Proteins in the Mutated Plants on the AtGCS1 Gene

6.1 Materials and Methods

Preparation of the N-glycansfrom Arabidopsis thaliana seeds

Raw protein extracts have been obtained by grinding 100 mg A. thaliana seeds in 10 mL Hepes 50 mM buffer, pH 7.5 containing 2 mM sodium bisulfite and 0.1% SDS. The insoluble material has been eliminated through centrifugation and then the proteins have been precipitated by addition of two ethanol volumes at −20° C. The residue is then heated for 3 minutes in 2 mL 50 mM Tris HCl buffer pH 7.5 containing 0.1% SDS. After the solution is cooled, 0.1 U Endo H have been added and the solution has been incubated for 18 hours at 37° C. The N-glycans have then been purified by successive elutions on C18 columns (Bond Elut), AG 50W-X2 and Carbograph as previously described in the litterature (Bardor et al., 1999).

HPAE-PAD Chromatography

HPAE-PAD chromatographies have been performed on a Dionex DX500 apparatus provided with a GP50 pumping system, an ED40 detector and a PA1 Carbopac column (4.6×250 mm). The N-glycans have been eluted using a 60 minute linear gradient ranging from 0 to 200 mM of sodium acetate in 100 mM soda.

MALDI-TOF Mass Spectrometry

The MALDI-TOF mass spectra have been recorded on a spec E Tof Micromass apparatus. The spectra have been performed in a positive and reflectron mode with a 20 kV acceleration voltage, a 10⁻⁷ mbar pressure in the source and a 10⁻⁶ mbar pressure in the analyzer. The nitrogen laser has been set at 337 nm with a pulse duration of 4 ns. The apparatus has been calibrated with substance P (1347.7 Da) and the human adrenocorticotropic hormone (2465.2 Da). The solution containing the sample has been prepared at an approximative concentration of 10 pmole.μL⁻¹ in water. Two μL of such a solution have been dissolved in the same volume of a matrix solution obained through dissolution of 2 mg of 5,5-dihydroxybenzoic acid in 200 μL acetonitrile at 70% containing 0.1% TFA. The matrix sample mixture has then been homogenized, then deposited on the target and vacuum dried.

Abbreviations: Endo H, endoglycosidase F/HPAEC-PAD, High pH Anion Exchange Chromatography with Pulsed Amperometric Detection/MALDI-TOF, Matrix-Assisted Laser Desorption Ionization-Time of Flight/

6.2 Results

The N-glycans have been released from the seed protein extracts by Endo H treatment. Such an endoglycosidase specifically cleaves the glycosidic link between the two GlcNAc's of the chitobiose unit of the oligomannosidic glycans. The HPAE-PAD chromatographic profile of the N-glycans released from wild seeds (FIG. 2A) exhibits six major peaks. Such peaks have been attributed to the Man₅GlcNAc to Man₉GlcNAc structures (see table I) by comparison of their retention time with standard structures as previously described in the litterature (Rayon et al., 1996). Such oligomannosidic structures have been previously characterized from wild Arabidosis plants by Rayon et al. (1999). The structures of these compounds have been able to be confirmed by MALDI-TOF mass spectrometry analysis (FIG. 3A). The spectrum exhibits five ions (M+Na⁺) at m/z=1 054, 1 216, 1 378, 1 540 and 1 702 corresponding to the expected masses for the sodium adducts of the Man₅GlcNAc to Man₉GlcNAc oligosaccharides.

The N-glycans associated to the mutant seed proteins have been analyzed following the same principle from the mixture of homozygous and heterozygous seeds. The HPAE-PAD profile (FIG. 2B) shows a peak set between 16 and 22 minutes, similar to those detected from wild seeds (FIG. 2A). Such peaks have been attributed to the Man₅GlcNAc to Man₉GlcNAc oligomannosidic N-glycans. Beside these structures, a 29 minute peak has been detected. The nature of the oligosaccharide(s) contained in this peak has first been studied by comparison of its HPAE-PAD retention time with compounds of known structures. It has been established (not shown) that the peak eluted at 29 minutes exhibits the same retention time as the Glc₃Man₇GlcNAc structure (Table I) isolated in the laboratory during a previous study on the effect of the castanospermine, inhibiting the α-glucosidase I, on the N-glycan maturation in sycamore cell cultures (Lerouge et al., 1996). The MALDI-TOF spectrum of the isolated N-glycans of mutant seeds (FIG. 3B) also shows the presence of additional structures to those identified from wild seeds. Beside (M+Na⁺) ions at m/z =1 054, 1 216, 1 378, 1 540 and 1 702 corresponding to the Man₅GlcNAc to Man₉GlcNAc species, two ions at m/z=1 864 and 2 026 have been detected. Such molecular ions correspond to structures having one or two additional hexoses. In order to confirm that such two ions are to be attributed to the additional peak observed in the profile shown in FIG. 2B, this peak has been collected, desalted on a carbograph column (Parker et al.,. 1998) then MALDI-TOF spectrometry analyzed. Only the ions at m/z=1 864 and 2 026 have been detected thereby confirming that the chromatographic peak at 29 minutes (FIG. 2) originates from the coelution of two oligosaccharides comprising a N-acetylglucosamine residue and respectively ten and eleven hexose residues. Such data allow to suggest that in the mutant seeds, two oligosaccharides, not observed in the wild seeds, are accumulated and have Glc₃Man₇GlcNAc and Glc₃Man₈GlcNAc structures (table I). The first structure has previously been characterized from sycamore cells after castanospermine treatment (Lerouge et al., 1996), the second structure carries an additional mannose residue probably resulting from the α-mannosidase 1 partial action at the level of the Golgi apparatus.

TABLE I
Structures and designations of the oligosaccharides

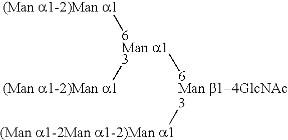

Man₅GlcNAc to Man₉GlcNAc

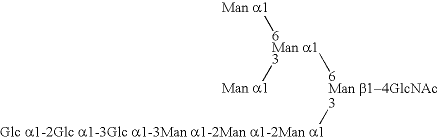

GIc₃Man₇GlcNAc

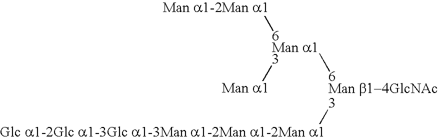

Glc₃Man₈GlcNAc

### BIBLIOGRAPHIC REFERENCES

ALTSCHUL, S. F., Gish, W, Miller, W., Myers, E. W., and Lipman, D. 1990. Basic local alignment search tool. J. Mol. Biol. 215, 403-410.
ALTSHUL S. F et al., (1997), Nucleic Acids Research, vol. 25:33896-3402.
AUSUBEL et al., (1989), Current Protocols in Molecular Biology, Green Publishing Associates and WLILEY Interscience, New-York.
BAUSE, E. SCHWEDEN, J., GROSS, A. AND ORTHEN, B. (1989) Purification and characterization of trimming glucosidase 1 from pig liver. Eur. J. Biochem. 183:661-669.
BAUSE, E., ERKENS, R., SCHWEDEN, J. AND JAENIK, L. (1986) Purification and characterization of trimming glucosidase 1 from Saccharomyces cerevisiae. FEBS 206(2):206-212.
BECHTOLD, N. ELLIS, J. PELLETIER, G. (1993). In planta Agrobacterium mediated gene transfer by infiltration of adult Arabidopsis thaliana plants, C.R. Acad. Sci. Paris, 316: 1194-1199.
BOUCHEZ, D. CAMILLERI, C. CABOCHE, M. (1993). A binary vector based on Basta resistance for in planta transformation of Arabidopsis thaliana. C.R. Acad. Sci. Paris. 316: 1188-1193.
BEAUCAGE et al., (1981), Tetrahedron Lett., vol. 22:1859-1862
BROWN EL. et al., (1979) Methods Enzymol., 68:109-151.
BEVAN et al., (1984) Nucleic Acids Research, 12:8711-8721.
BOUCHEZ et al., 1993, C.R. Acad. Sci. Paris, Science de la Vie/Life Sciences, 316:1188-1193.
BARDOR M., LOUTELIER-BOURHIS C., MARVIN L., CABANES-MACHETEAU M, LANGE C., LEROUGE P. AND FAYE L. (1999) Analysis of plant glycoproteins by Matrix-Assisted Laser Desorption onization Mass Spectrometry: Application to the N-glycosylation analysis of bean phytohemagglutinin. Plant Physiol. Biochem., 37, 319-325.
CHO R. J. et al. (1998), Proc. Natl. Acad. Sci. USA, Vol.95 (7):3752
CORMACK B, VALDIVIA R. H. FALKOW S. 1996. Gene 173, 33-38.
CHRISPEELS, M. ET FAYE, L. (1998) Rien ne sert de produire, il faut glycosyler. Biofutur, 184: 53-57.
DEVIC, M. ALBERT, S. DELSENY, M., ROSCOE, J. (1997). Efficient PCR walking on plant genomic DNA. Plant Physiol. Biochem. 35 (4): 331-339.
FULLER S. A. et al. (1996), Immunology in Current Protocols in Molecular Biology.
JEFFERSON R. A., KAVANACH, T. A., and BEVAN, M. W. (1987). Embo J. 3901-3907.
FICHETTE-LAINÉ et al., 1998, Methods in Biotechnology, 3:271-290
GREEN et al.,(1986), Ann. Rev. Biochem., vol. 55:569-597.
GRIFFIN et al. (1989), Science, vol. 245:967-971.
GARCIA-CASADO, G. SANCHEZ-MONGE, R. CHRISPEELS, M. ARMANTIA, A., SALCEDO, G. AND GOMEZ, L. (1996) Role of complex asparagine-linked glycans in the allergenicity of plant glycoprotein. Glycobiology, 6(4): 471-477.
GOMORD, V., FITCHETTE-LAINÉ, A-C., DENMAT, L-A., MICHAUD, D. AND FAYE, L. (1998) Production of Foreign Proteins in Tobacco Cell Suspension Culture. Methods in Biotechnology. Cunningam, C and Porter, A (Eds) Humana Press Inc, Totwa, N.J. 3 : 15.
HAMES B. D. and HIGGINS S. J., (1985), Nucleic Acid Hybridization: The Practical Approach, HANES and HIGGINS Ed., IRL Press, Oxford.
HAQ, T., MANSON, H., CLEMENTS, J. AND ARNTZEN, C. (1995) Oral Immunization with a recombinant bacterial antigen produced in transgenic plants. Science, 268 (5211): 714-716.
HETTKAMP, H., LEGLER, G. AND BAUSE, E. (1984). Purification by affinity chromatography of glucosidase 1, an endoplasmic reticulum hydrolase involved in the processing of asparagine-linked oligosaccharides. Eur. J. Biochem. 142(1) : 85-90.
HOHN AND PUCHTA, 1999, Gene Therapy in Plant, Proc. Natl. Acad. Sci. USA, 96:8321-8323
HOUBEN WEYL, (1974), In Methoder Organischen Chemie, E.Wunsch Ed. vol. 15-I et 15-II, Thieme, Stuttgart.
HORSCH et al., (1984).
IZANT J J, WEINTRAUB H., (1984), Cell, Vol. 36 (4):1007-1015
KOCH Y. (1977), Biochem. Biophys. Res. Commun. Vol. 74:488-491.
KOHLER G. and MILSTEIN C., (1975) Nature, vol. 256:495.
KOZBOR et al. (1983), Hybridoma, vol.2 (1) : 7-16.
KALZ-FÜLLER, B., BIEBERICH, E. AND BAUSE, E. (1995). Cloning and expression of glucosidase 1 from human hippocampus. Eur. J. Biochem 231: 344-351.
KHAN, F., Varma, G. and Vijay, I. (1999). Genomic organisation and promoter activity of glucosidase 1 gene. Glycobiology, 9 (8): 797-806.
LEROUGE, P., CABANES-MACHETEAU, M., RAYON, C., FISCHETTE-LAINÉ, A-C, GOMORD, V. AND FAYE, L. (1998) N-Glycoprotein biosynthesis in plants : recent developments and future trends. Plant Molecular Biology, 38 : 31-48.
LEROUGE P., FITCHETTE-LAINÉ A. C., CHEKKAFI A., AVIDGOR V. AND FAYE L. (1996). Castanospermine inhibits the processing of N-glycans in sycamore cells without affecting the secretion of glycoproteins. Plant J., 10, 713-719.
MARTINEAU et al., JOHNS P. WINTER G.(1998), J. Mol. Biol., vol. 280 (1):117-127.
MERRIFIELD R B, (1965a), Nature, vol. 207 (1996):522 -523)
MERRIFIELD R. B., (1965b), Science, vol. 150 (693):178-185.
MYLNE, J., and BOTELLA, J., R. (1998). Binary vectors for sense and antisense expression of Arabidopsis ESTs. Plant Molecular. Biology Reporter 16, p 257-262.
McELROY D., ZHANG W, CAO J, WU R (1990) Isolation of an efficient actin promoter for use in rice transformation. Plant Cell 2, p 163-171.
MANSFIELD, S. AND BRIARTY, L. 1991, Early embryogenesis in Arabidopsis thaliana II. The Developing embryo; Can. J. Biol., 69:461-476.
MURRAY ET AL. 1987, Lectin-specific targeting of lysosomal enzymes to reticuloendothelial cells. Methods Enzymol; 149:25-42.
NARANG S. A. et al., (1979), Methods Enzymol., vol. 68, 90-98.
ODELL J T, NAGY F. CHUA N H. (1985). Identification of DNA sequences required for activity of the cauliflower mosaic virus 35S promoter. Nature 313, p. 810-812.
PALACPAC, N., Yoshida, S. Sakai, H., Kimura, Y., Fujiyama, K. Yoshid, T. and Seki, T. (1999) Stable expression of human β-1,4-galactosyltransferase in plant cell modified N-linked glycosyltion patterns. Proc. Natl. Acad. Sci :. USA, 96:4692-4697.
PACKER H. N., MARGARET A. L., JARDINE D. R., REDMOND J. W. (1998) A general approach to desalting oligosaccharides released from glycoproteins, Glycoconjugate J., 15, 563-570.
PUKAZHENTHI, B., VARMA, G. AND VIJAY, I. (1993) Conserved structural features in glycoprotein processing glucosidase 1 from several tissues and species. Indian J Biochem Biophys. 30(6):333-40.
ROSSI et al. (1991), Pharmacol. Ther., vol. 50 :245-254
RIDDER R. et al., (1995), Biotechnology (NY), vol. 13 (3):255-260 ou encore des anticorps humanisés
REINMANN K. A. et al., (1997), Aids, Res. Hum. Retroviruses, vol. 13 (11):933-943
RAYON R., GOMORD V., FAYE L., LEROUGE P. (1996). N-glycosylation of phytohemagglutinin expressed in bean cotyledons or in transgenic tobacco plants, Plant Physiol. Biochem., 34, 273-281.
RAYON C., CABANES-MACHETEAU M., LOUTELIER-BOURHIS C., SALIOT-MAIRE I., LEMOINE J., REITER W. D., LEROUGE P. AND FAYE L. (1999) Characterization of N-glycans from Arabidopsis thaliana. Application to a fucose-deficient mutant. Plant Physiol., 119, 725-733.
ROMANIOUK, A. AND VIJAY, I. (1997) Structure-function relashionships in glucosidase I: amino-acids invoved in binding the substrate to the enzyme. Glycobiology. 7(3) 399-404.
SHAILUBHAI, K. PRATTA, M. AND VIJAY, I (1987). Purification and characterization of glucosidase 1 involved in N-linked glycoprotein processing in bovine mammary gland. Biochem. J. 247 (3):555-562.
SHAILUBRAI, K. PUKAZHENTI, B. SEXENA, E., VARMA, G. AND VIJAY, I. (1991) Glucosidase 1, a transmembrane endoplasmic reticular glycoprotein with a luminal catalytic domain. J. Biol. Chem. 266(25): 16587-93.
SAMBROOK J., FRITSCH E. F. and T. MANIATIS, (1989), Molecular Cloning: A Laboratory Manual 2ème ed. Cold Spring Harbor Laboratory, Cold Spring Harbor N.Y.
SCZAKIEL G. et al. (1995) Trends. Microbiol., vol. 3 (6) :213-217.
SANCHEZ-PESCADOR R., (1988), J. Clin. Microbiol., vol. 26 (10):1934, 1938.
SCHUTZE, M., PETERSON, P., AND JACKSON, M. (1994) An N-terminal double arginine motif maintains type II membrane proteins in the endoplasmic reticulum. EMBO Journal 13(7) :1696-1705.
SZUMILO, T. KAUSHAL, G. AND ELBEIN, A. (1986). Purification and properties of Glucosidase 1 from Mung Bean Seddlings. Archives of Biochemistry and Biophysics (247):261-271.
URDEA N S et al. (1988), Nucleic Acids Research, vol. 11:4937-4957.
URDEA N S et al., (1991), Nucleic Acids Symp. Ser., vol. 24:197-200.
WEIL, J. H. BONNET, J. BOULANGER, Y., CHAMBON, P. DUBERTET, G. GAUTHERON, D. KEDINGER, C., LADZUNSKI, M., MONTREUIL, J., PATTE, J. C., REBEL, G., ROSSIGNOL, J. M. E T WRIGHT, M. (1989) Biochimie Générale, 5^{ème} édition. Eds Masson, PARIS.
WABIKO H. et al., (1986), DBA, vol. 5 (4):305-314.
WOO, S.-S, Jiang J., Gill, B. S., Paterson, A. H. and Wing, R. A. 1994. Nuc. Acid Res. 22, p 4922-4931.
ZENG, Y-C. AND ELBEIN, A. (1998) Purification to homogeneity and properties of plant glucosidase I. Arch Biochem Biophys. 355(1):26-34.
ZENG ET AL, 1997: ZENG Y, BANNON G, THOMAS V., RICE K., DRAKE R AND ELBEIN, A. (1997) Purification and Specificity of 1,2-Xylosyltransferase, an Enzyme That Contributes to the Allergenicity of Some Plant Proteins. J. Biol. Chem. 272 (50):31340-31347.

1. A nucleic acid comprising at least 20 consecutive nucleotides of a polynucleotide coding for a type I glucosidase having the SEQ ID N^{o} 1 aminoacid sequence, or a nucleic acid with a complementary sequence.

2. A nucleic acid according to claim 1, comprising at least 20 consecutive nucleotides having the SEQ ID N^{o} 2 nucleotidic sequence, or a nucleic acid with a complementary sequence.

3-47. (canceled)


SEQUENCE LISTING (15 sequences)

SEQ ID NO: 1 - protein, 852 aa, Arabidopsis thaliana
MTGASRRSARGRIKSSSLSPGSDEGSAYPPSIRRGKGKELVSIGAFKTNLKILVGLIILGIIVIYFVINRLVRHGLLFDESQKPRVITPFPAPKVMDLSMFQGEHKESLYWGTYRPHVYFGVRARTPLSLVAGLMWLGVKDEMYVMRHFCENSDDLSTFGWREHNGRDYGRQELVENDMVIETSFVKSKGDGLGYGGDWAVRIDVKNKGLNDDVKRSAHLFFYLADEGGNVLNLGQDGLDFQGSSLLVSGSREDVGDWQIHLKSQNQLETHYSGFKTPHIYNLSDLVQQNLALQARKFGRLQLSDTSEDSSNIYIFQISGRLPFTIDIPFISGIKGESSNVEKRLTSLTGLPLSDLLKKKHQEFDAKFNECFNLSEKHDSETLGVGRTAIANMLGGIGYFYGQSKIYVPKSTQPGSRDNFLLYWPAELYTAVPSRPFFPRGFLWDEGFHQLLIWRWDIRITLDIVGHWLDLLNIDGWIPREQILGAEALSKVPEEFVVQYPSNGNPPTLFLVIRDLIDAIRMEKFVASEKDEVLSFLERASVRLDAWFQWFNTSQKGKEIGSYFWHGRDNTTTQELNPKTLSSGLDDYPRASHPSEDERHVDLRCWMYRAADCMHSITELLGKEDKLSKENYNSTAKLLSNFNLLNQMHYDSDYGAYFDFGNHTEKVKLIWKEVIQENGQLSRQLVRKTFGKPKLKLVPHLGYVSFFPFMSRIIPPDSPILEKQLNLISNRSILWSDYGLVSLAKTSSMYMKRNTEHDAPYWRGPIWMNMNYMILSSLYHYSIVDGPYREKSKAIYTELRSNLIRNVVRNYYETGYIWEQYDQVKGTGKGTRLFTGWSALTLLIMSEDYPIF

SEQ ID NO: 2 - DNA, 2559 bp, Arabidopsis thaliana
atgaccggagctagccgtcggagcgcgcgtggtcgaatcaaatcatcatcattatctcccggctccgatgaaggaagcgcttatcctccgagtatccgccgcggcaaaggcaaggaacttgtatcgatcggtgctttcaagacgaatctcaagatattggttgggttaatcatattagggattatagtaatctacttcgtaatcaatcggctagttcgtcacgggttgttgtttgatgagtcccagaagcctagggttatcactccttttcctgctccgaaagtcatggatttgtcaatgtttcagggcgagcacaaagagagcttatactggggaacttatcgtcctcatgtttattttggagttcgagctagaactccattgtccttggtagctggcttgatgtggcttggtgtcaaagatgagatgtatgttatgcggcatttctgtgaaaactctgatgatttaagtacatttggctggagagaacataatggacgggattatggtcggcaagagctggttgaaaacgatatggtaatagagacgagttttgtgaagtccaagggagacggccttggttatggtggggattgggcagttcggattgatgtaaaaaataaagggttgaatgatgatgtgaagagaagtgcacatctcttcttttatttagctgatgaaggtggcaatgttttaaatttaggccaagatggtttagatttccaagggagctctctcctggtttcggggtcacgtgaagatgtaggagactggcagatacacttaaaatcacagaatcagctcgagacacattattccggtttcaagacgcctcacatatataatttgtctgatctagttcagcaaaatcttgctctgcaggcaaggaagtttggacgacttcagctctctgacacatcagaggattcttctaacatttacatctttcagatttctgggaggctaccatttacgattgatattcccttcatctccgggatcaaaggagaaagttcaaatgtggaaaagcgtttaacaagtctcacaggtttgccactctctgatctacttaaaaagaaacatcaagaatttgatgcaaagtttaatgaatgcttcaacctttctgaaaagcatgattcagaaacattgggggttggaaggactgcaattgcaaacatgcttgggggcattggttacttctatggccaatcaaaaatttatgttcctaaaagcactcagcctggaagtcgggataatttcttgctgtattggccagccgagctgtacacagctgttccaagccgacctttttttcctagggggtttttgtgggacgaaggtttccatcaattgttgatctggcggtgggatattcgtataaccttggatatcgttggacactggttagacctattgaacatagatggatggattccacgtgagcaaattttgggtgctgaagctttgagtaaggttccagaggaattcgtagttcaatacccgagtaatggcaatccaccaactctgtttttggtgatacgtgatctgattgacgctataaggatggaaaagtttgtcgcatcagaaaaggatgaagtattgtcattccttgagcgagcttctgtacgcttagatgcttggtttcagtggtttaatacttcccagaaaggcaaggagatcggaagctacttttggcatggcagagacaacacaacaactcaagaacttaaccctaagacgctttcgtcggggctggatgattatccccgggcttcgcaccctagtgaagatgagcgacatgttgatcttagatgctggatgtaccgtgctgcagattgcatgcattccatcacagagcttttaggaaaagaagacaaactttcaaaggagaattacaattcaactgccaagctgctttcaaatttcaatcttctgaatcagatgcactatgatagtgactatggggcttactttgactttggcaatcatacagaaaaagttaagttgatatggaaagaggtaattcaagaaaatggtcaattatctcgacaacttgtaaggaagacttttgggaagccaaagctaaaattggttcctcacttgggttacgtcagcttctttcccttcatgtctagaatcatcccacctgattctccaattctcgagaaacagctcaatctcatttcaaataggagcatcttatggagtgactatggactagtttcgcttgccaaaaccagttctatgtatatgaaacgtaacactgagcatgatgcaccatactggagaggtcctatatggatgaacatgaactacatgattctttcttctctttaccattactctatagtggatggaccgtatagagaaaagtcgaaagcaatttacacggaactaaggagcaatttgataaggaacgtggttagaaactattacgagacggggtacatctgggaacaatatgatcaagttaagggaacaggcaaaggcacgcgtctcttcaccggctggtctgcgcttaccttgttaattatgtcagaagattatcctattttttag

SEQ ID NO: 3 - DNA, 20 bp, Arabidopsis thaliana
atgaccggagctagccgtcg

SEQ ID NO: 4 - DNA, 20 bp, Arabidopsis thaliana
aagtttcgttcccgaagagg

SEQ ID NO: 5 - DNA, 43 bp, Arabidopsis thaliana
ctaatacgactcactatagggctcgagcggccgcccggggagt

SEQ ID NO: 6 - DNA, 8 bp, Arabidopsis thaliana
  misc_feature (1): Phosphoric group (PO4) linked in the 5′ position of the sugar
acctcccc

SEQ ID NO: 7 - DNA, 27 bp, Arabidopsis thaliana
ggatcctaatacgactcactatagggc

SEQ ID NO: 8 - DNA, 18 bp, Arabidopsis thaliana
ctatagggctcgagcggc

SEQ ID NO: 9 - DNA, 16 bp, Arabidopsis thaliana
ggccgcccggggaggt

SEQ ID NO: 10 - DNA, 23 bp, Arabidopsis thaliana
tcgttaaaactgcctggcacagc

SEQ ID NO: 11 - DNA, 25 bp, Arabidopsis thaliana
ccagactgaatgcccacagcccgtc

SEQ ID NO: 12 - DNA, 24 bp, Arabidopsis thaliana
tcacgggttggggttctacaggac

SEQ ID NO: 13 - DNA, 24 bp, Arabidopsis thaliana
cggctattggtaataggacactgg

SEQ ID NO: 14 - DNA, 26 bp, Arabidopsis thaliana
caaccctcaactggaaacgggccgga

SEQ ID NO: 15 - DNA, 26 bp, Arabidopsis thaliana
cgtgtgccaggtgcccacggaatagt